(19) United States
(12) Patent Application Publication
(10) Pub. No.: US 2008/0279769 A1
(43) Pub. Date: Nov. 13, 2008

(54) Enzyme Inhibitor Imaging Agents

(76) Inventors: Magne Solbakken, Oslo (NO); Alan Cuthbertson, Oslo (NO); Anthony Eamon Storey, Buckinghamshire (GB); Alexander Jackson, Buckinghamshire (GB); Sally-Ann Ricketts, Buckinghanshire (GB); Peter Brian Iveson, Buckinghamshire (GB)

Correspondence Address: GE HEALTHCARE, INC., IP DEPARTMENT, 101 CARNEGIE CENTER, PRINCETON, NJ, 08540-6231, US

(21) Appl. No.: 11/575,977

(22) PCT Filed: Sep. 23, 2005
(86) PCT No.: PCT/GB2005/003679

§ 371 Date: Feb. 13, 2008

(30) Foreign Application Priority Data
Sep. 24, 2004 (GB) 0421308.8

(51) Int. Cl. A61K 51/04; C07C 233/47; C07D 209/20; A61K 31/404; A61K 31/16

(52) U.S. Cl. 424/1.65; 564/153; 548/495; 514/616; 514/415

(57) ABSTRACT

The present invention discloses that imaging agents which comprise a specific type of matrix metalloproteinase inhibitors (MMPi's) of the hydroxamate class labelled with an imaging moiety, are useful diagnostic imaging agents for in vivo imaging and diagnosis of the mammalian body.

### FIELD OF THE INVENTION

The present invention relates to diagnostic imaging agents for in vivo imaging. The imaging agents comprise a metalloproteinase inhibitor labelled with an imaging moiety suitable for diagnostic imaging in vivo.

### BACKGROUND TO THE INVENTION

The matrix metalloproteinases (MMPs) are a family of at least 20 zinc-dependent endopeptidase enzymes which mediate degradation, or remodelling of the extracellular matrix (ECM) [Massova et al FASEB J 12 1075 (1998)]. Together, the members of the MMP family can degrade all of the components of the blood vessel wall and therefore play a major role in both physiological and pathological events that involve the degradation of components of the ECM. Since the MMPs can interfere with the cell-matrix interactions that control cell behaviour, their activity affects processes as diverse as cellular differentiation, migration, proliferation and apoptosis. The negative regulatory controls that finely regulate MMP activity in physiological situations do not always function as they should. Inappropriate expression of MMP activity is thought to constitute part of the pathological mechanism in several disease states. MMPs are therefore targets for therapeutic metalloproteinase inhibitors (MMPi's) in many inflammatory, malignant and degenerative diseases [Whittaker et al Chem. Rev. 99, 2735 (1999)].

Consequently, it is believed that synthetic inhibitors of MMPs may be useful in the treatment of many inflammatory, malignant and degenerative diseases. Furthermore, it has been suggested that inhibitors of MMPs may be useful in the diagnosis of these diseases. U.S. Pat. No. 5,183,900 discloses compounds for the treatment of diseases associated with MMPs, the compounds of formula:

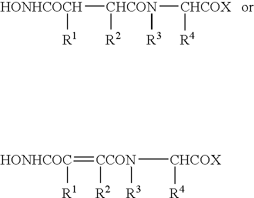

where R¹ is H and R² is alkyl (3-8C) or wherein R¹ and R² taken together are —(CH₂)ₙ— wherein n=3-5; R³ is H or alkyl (1-4C); R⁴ is fused or conjugated unsubstituted or substituted bicycloaryl methylene; X is OR⁵ or NHR⁵, wherein R⁵ is H or substituted or unsubstituted alkyl(1-12C), aryl (6-12C), aryl alkyl (6-16C); or X is an amino acid residue or amide thereof; or X is the residue of a cyclic amine or heterocyclic amine. U.S. Pat. No. 5,183,900 states that the compounds can be labelled with scintigraphic labels such as ⁹⁹Tc or ¹³¹I, to determine the location of excess amounts of MMPs in vivo, but does not teach or suggest how such labelling is achieved.

WO 01/60416 discloses chelator conjugates of a wide range of different classes of matrix metalloproteinase (MMP) inhibitors, and their use in the preparation of metal complexes with diagnostic metals. The MMP inhibitors described include hydroxamates, including some succinyl hydroxamates (as described on page 86 line 30 to page 89 line 9). The compounds are proposed to be useful in the diagnosis of cardiovascular pathologies associated with extracellular matrix degradation such as atherosclerosis, heart failure and restenosis. Preferred MMP inhibitors, chelators and linkers are described therein. A report by Zheng et al [Nucl. Med. Biol. 29 761-770 (2002)] documented the synthesis of hydroxamate MMP inhibitors labelled with the positron emission tomography (PET) tracers ¹¹C and ¹⁸F. The compounds described therein are postulated to be useful in the non-invasive imaging of breast cancer.

### THE PRESENT INVENTION

It has now been found that a particular class of succinyl hydroxamate matrix metalloproteinase inhibitors (MMPi's) labelled with an imaging moiety are useful diagnostic imaging agents for in vivo imaging and diagnosis of the mammalian body. These compounds present superior MMP inhibitory activity with Ki in the sub-nanomolar range vs Gelatinases (MMP-2 and MMP-9) and Collagenases (MMP-1, MMP-8 and MMP-13). The urinary excretion profiles of the MMPi's of the invention can be adjusted by use of appropriate linker groups, especially polyethyleneglycol (PEG), amino acid or sugar-containing linker groups.

The imaging agents of the present invention are useful for the in vivo diagnostic imaging of a range of disease states (inflammatory, malignant and degenerative diseases) where specific matrix metalloproteinases are known to be involved. These include:
(a) atherosclerosis, where various MMPs are overexpressed. Elevated levels of MMP-1, 3, 7, 9, 11, 12, 13 and MT1-MMP have been detected in human atherosclerotic plaques [S. J. George, Exp. Opin. Invest. Drugs, 9(5), 993-1007 (2000) and references therein]. Expression of MMP-2 [Z. Li et al, Am. J. Pathol., 148, 121-128 (1996)] and MMP-8 [M. P. Herman et al, Circulation, 104, 1899-1904 (2001)] in human atheroma has also been reported. The collagenases are believed particularly important to VPs, Circulation, 1999, 99, 2503, Sukhova et. al.; ibid, 2001, 104, 1899, Herman et. al. referenced above; Stroke, 2002, 33, 2858, Axisa et. al.; DDT, 2002, 7, 86, Fricker; C];
(b) chronic heart failure (Peterson, J. T. et al. Matrix metalloproteinase inhibitor development for the treatment of heart failure, Drug Dev. Res. (2002), 55(1), 29-44 reports that MMP-1, MMP-2, MMP-3, MMP-8, MMP-9, MP-13 and MMP-14 are upregulated in heart failure);
(c) cancer [Vihinen et al, Int. J. Cancer 99, p 157-166 (2002) reviews MMP involvement in cancers, and particularly highlights MMP-2, MMP-3, MMP-7, and MMP-9];
(d) arthritis [Jacson et al, Inflamm. Res. 50(4), p 183-186 (2001) “Selective matrix metalloproteinase inhibition in rheumatoid arthritis-targeting gelatinase A activation”, MMP-2 is particularly discussed];
(e) amyotrophic lateral sclerosis [Lim et al, J. Neurochem, 67, 251-259 (1996); where MMP-2 and MMP-9 are involved];
(f) brain metastases, where MMP-2, MMP-9 and MMP-13 have been reported to be implicated [Spinale, Circul. Res., 90, 520-530 (2002)];
(g) cerebrovascular diseases, where MMP-2 and MMP-9 have been reported to be involved [Lukes et al, Mol. Neurobiol., 19, 267-284 (1999)];
(h) Alzheimer's disease, where MMP-2 and MMP-9 have been identified in diseased tissue [Backstrom et al, J. Neurochem., 58, 983-992 (1992)];
(i) neuroinflammatory disease, where MMP-2, MMP-3 and MMP-9 are involved [Mun-Bryce et al, Brain. Res., 933, 42-49 (2002)];
(j) COPD (ie. chronic obstructive pulmonary disease) where MMP-1, MMP-2, MMP-8 and MMP-9 have been reported to be upregulated [Segura-Valdez et al, Chest, 117, 684-694 (2000)] amongst others;
(k) eye pathology [Kurpakus-Wheater et al, Prog. Histo. Cytochem., 36(3), 179-259 (2001)];
(l) skin diseases [Herouy, Y., Int. J. Mol. Med., 7(1), 3-12 (2001)].

The succinyl hydroxamate MMP is of the present invention are more hydrophilic than alternative MMP is of comparable potency. They exhibit superior clearance from background tissues in vivo, and are available via a flexible synthetic route, which permits the facile incorporation of a range of imaging moieties.

### DETAILED DESCRIPTION OF THE INVENTION

In a first aspect, the present invention provides an imaging agent which comprises a metalloproteinase inhibitor of Formula (I) labelled with an imaging moiety at position X¹, X², X³, X⁴ or Y¹, wherein the imaging moiety can be detected following administration of said labelled matrix metalloproteinase inhibitor to the mammalian body in vivo

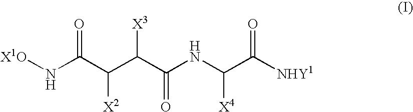

where:
X¹ is H, C₁₋₃ alkyl or C₁₋₃ fluoroalkyl;
X² is H, C₁₋₆ alkyl, C₃₋₆ cycloalkyl or C₁₋₆ fluoroalkyl;
X³ is an X² group, NH₂, C₁₋₁₀ amino or —NH(CO)X^{a} where X^{a} is C₁₋₆ alkyl, C₃₋₁₂ aryl or C₅₋₁₅ aralkyl;
X⁴ is C₁₋₆ alkyl, Ar¹ or —(C₁₋₃ alkyl)Ar¹, where Ar¹ is a C₃₋₁₂ aryl or heteroaryl group or —(CH₂)_{w}CONHY², where w is an integer of value 1 or 2;
Y¹ and Y² are independently Y groups, where Y is C₁₋₁₀ alkyl, C₃₋₁₀ cycloalkyl, C₁₋₁₀ fluoroalkyl, an Ar¹ group or —(C₁₋₃ alkyl)Ar¹;
with the provisos that:
(i) X² and X³ are not both H;
(ii) when X¹ is H, X² is H or C₁₋₃ alkyl and X³ is C₁₋₆ alkyl, C₃₋₆ cycloalkyl or C₁₋₆ fluoroalkyl, and X⁴ is C₁₋₆ alkyl, phenyl or benzyl, the imaging moiety does not comprise a chelating agent.

In Formula (I), X¹ is most preferably H.

X² is preferably H, C₁₋₄ alkyl or C₁₋₄ fluoroalkyl, and is most preferably H, C₂₋₄ alkyl or C₂₋₄ fluoroalkyl, with X² equal to —CH₂CH(CH₃)₂ being most especially preferred.

When X³ is an X² group, it is preferably H, C₁₋₄ alkyl or C₁₋₄ fluoroalkyl, and is most preferably H, C₂₋₄ alkyl or C₂₋₄ fluoroalkyl. When X³ comprises an amine group, it preferably comprises a primary amine group such as —NH₂ or —(CH₂)_{q}NH₂ where q is an integer of value 1 to 4, to permit facile conjugation of the imaging moiety at that position (eg. by reductive amination or N-alkylation). A further preferred amine-containing X³ group is —NH(C₁₋₄ alkyl), especially —NHCH(CH₃)₂ which is a N-containing analogue of a —CH₂CH(CH₃)₂ group. The most preferred compounds of Formula (I) are where X³ is an X² group.

In Formula (I), X² and X³ are not both H, ie. substituents at both the X² and X³ positions are within the scope of the present invention. A preferred combination is that one of X² and X³ is H, and the other is not H. For this combination, it is especially preferred that one of X² and X³ is H, and the other is —CH₂CH(CH₃)₂. The present inventors have found that, surprisingly, substitution at the X² position, gives potent MMP inhibitors. Hence, a most preferred combination is that X³ is H when X² is a preferred X² group as defined above, with X¹ equal to H. Most especially preferably, X¹ and X³ are both H and X² is —CH₂CH(CH₃)₂.

X⁴ is preferably —CH₂Ar¹ or —(CH₂)CONHY². When X⁴ is —CH₂Ar¹, Ar¹ most preferably comprises an indolyl group, especially —CH₂(3-indolyl), ie.

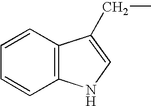

Y¹ is preferably C₁₋₁₀ alkyl, C₁₋₁₀ fluoroalkyl or —(CH₂)_{w}CONHY², most preferably C₁₋₄ alkyl, C₁₋₄ fluoroalkyl or —(CH₂)CONHY², with Y¹ equal to —CH₃ or —(CH₂)CONHAr¹ being especially preferred.

The hydroxamate matrix metalloproteinase inhibitors of the present invention is suitably of molecular weight 100 to 3000 Daltons, preferably of molecular weight 150 to 600 Daltons, and most preferably of molecular weight 200 to 500 Daltons. The inhibitor is preferably of synthetic origin.

The term “labelled with” means that the MMPi itself either comprises the imaging moiety, or the imaging moiety is attached as an additional species, optionally via a linker group, as described for Formula II below. When the MMPi itself comprises the imaging moiety, this means that the ‘imaging moiety’ forms part of the chemical structure of the MMPi and is a radioactive or non-radioactive isotope present at a level significantly above the natural abundance level of said isotope. Such elevated or enriched levels of isotope are suitably at least 5 times, preferably at least 10 times, most preferably at least 20 times; and ideally either at least 50 times the natural abundance level of the isotope in question, or present at a level where the level of enrichment of the isotope in question is 90 to 100%. Examples of MMPi's comprising the ‘imaging moiety’ are described below, but include CH₃ groups with elevated levels of ¹³C or ¹¹C and fluoroalkyl groups with elevated levels of ¹⁸F, such that the imaging moiety is the isotopically labelled ¹³C, ¹¹C or ¹⁸F within the chemical structure of the MMPi. The “imaging moiety” may be detected either external to the mammalian body or via use of detectors designed for use in vivo, such as intravascular radiation or optical detectors such as endoscopes, or radiation detectors designed for intra-operative use. Preferred imaging moieties are those which can be detected externally in a non-invasive manner following administration in vivo. Most preferred imaging moieties are radioactive, especially radioactive metal ions, gamma-emitting radioactive halogens and positron-emitting radioactive non-metals, particularly those suitable for imaging using SPECT or PET.

The “imaging moiety” is preferably chosen from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging;
(vii) a β-emitter suitable for intravascular detection.

When the imaging moiety is a radioactive metal ion, ie. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ⁹⁹Tc, ⁶⁴Cu, ⁶⁸Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹³¹I or ⁷⁷Br. A preferred gamma-emitting radioactive halogen is ¹²³I.

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N, ¹⁸F and ¹²⁴I, especially ¹¹C and ¹⁸F, most especially ¹⁸F.

When the imaging moiety is a hyperpolarised NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarised” is meant enhancement of the degree of polarisation of the NMR-active nucleus over its' equilibrium polarisation. The natural abundance of ¹³C (relative to ¹²C) is about 1%, and suitable ¹³C-labelled compounds are suitably enriched to an abundance of at least 5%, preferably at least 50%, most preferably at least 90% before being hyperpolarised. At least one carbon atom of the metalloproteinase inhibitor of the present invention is suitably enriched with ¹³C, which is subsequently hyperpolarised.

When the imaging moiety is a reporter suitable for in vivo optical imaging, the reporter is any moiety capable of detection either directly or indirectly in an optical imaging procedure. The reporter might be a light scatterer (eg. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, eg. cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared region, between 400 nm and 3 μm, particularly between 600 and 1300 nm. Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

When the imaging moiety is a β-emitter suitable for intravascular detection, suitable such β-emitters include the radiometals ⁶⁷Cu, ⁸⁹Sr, ⁹⁰Y, ¹⁵³Sm, ¹⁸⁶Re, ¹⁸⁸Re or ¹⁹²Ir, and the non-metals ³²P, ³³P, ³⁸S, ³⁸Cl, ³⁹Cl, ⁸²Br and ⁸³Br.

The MMPi of the present invention will possess chiral centres at the carbon atoms bearing the X⁴, plus X² and/or X³ substituents, plus possibly at other positions. The present invention encompasses all such stereoisomers in all degrees of purity, including racemic mixtures as well as substantially pure optical isomers (ie. enantiomers) or diastereomers. Preferred stereoisomers of Formula I are given below as Formulae Ia and Ib:

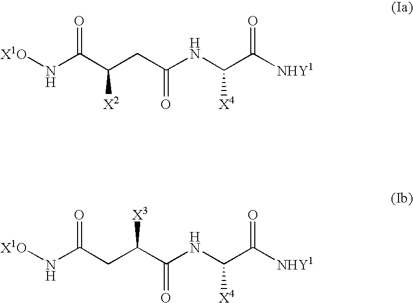

The imaging agents of the present invention are preferably of Formula II:

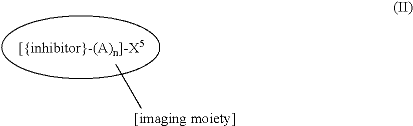

where:
   {inhibitor} is the metalloproteinase inhibitor of Formula (I);
   -(A)ₙ- is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
   R is independently chosen from H, C₁₋₄ alkyl, C₂₋₄ alkenyl, C₂₋₄ alkynyl, C₁₋₄ alkoxyalkyl or C₁₋₄ hydroxyalkyl;
   n is an integer of value 0 to 10; and
   X⁵ is H, OH, C₁₋₄ alkyl, C₁₋₄ alkoxy, C₁₋₄ alkoxyalkyl, C₁₋₄ hydroxyalkyl or an Ar¹ group as defined for Formula (I);
   the “imaging moiety” is as defined for Formula (I) above, and is attached at position X¹, X², X³, X⁴ or Y¹.

By the term “amino acid” is meant an L- or D-amino acid, amino acid analogue (eg. napthylalanine) or amino acid mimetic which may be naturally occurring or of purely synthetic origin, and may be optically pure, i.e. a single enantiomer and hence chiral, or a mixture of enantiomers. Preferably the amino acids of the present invention are optically pure.

By the term “sugar” is meant a mono-, di- or tri-saccharide. Suitable sugars include: glucose, galactose, maltose, mannose, and lactose. Optionally, the sugar may be functionalised to permit facile coupling to amino acids. Thus, eg. a glucosamine derivative of an amino acid can be conjugated to other amino acids via peptide bonds. The glucosamine derivative of asparagine (commercially available from Novabiochem) is one example of this:

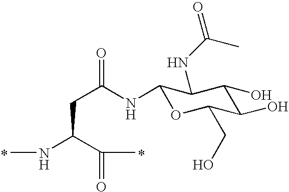

The imaging moiety is preferably attached at the X¹³, X⁴ or Y¹ positions of the MMPi of Formula (I), and is most preferably attached at the X⁴ or Y¹ positions, such that X¹ is H. It is especially preferred that the imaging moiety is attached to or comprises one of the Y² groups of a —(CH₂)_{w}(CO)NHY² moiety.

It is envisaged that the role of the linker group -(A)ₙ- of Formula II is to distance the imaging moiety from the active site of the metalloproteinase inhibitor. This is particularly important when the imaging moiety is relatively bulky (eg. a metal complex), so that binding of the inhibitor to the MMP enzyme is not impaired. This can be achieved by a combination of flexibility (eg. simple alkyl chains), so that the bulky group has the freedom to position itself away from the active site and/or rigidity such as a cycloalkyl or aryl spacer which orientates the metal complex away from the active site.

The nature of the linker group can also be used to modify the biodistribution of the imaging agent. Thus, eg. the introduction of ether groups in the linker will help to minimise plasma protein binding. When -(A)ₙ- comprises a polyethyleneglycol (PEG) building block or a peptide chain of 1 to 10 amino acid residues, the linker group may function to modify the pharmacokinetics and blood clearance rates of the imaging agent in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, eg. via the kidneys as opposed to via the liver.

When -(A)ₙ- comprises a peptide chain of 1 to 10 amino acid residues, the amino acid residues are preferably chosen from glycine, lysine, aspartic acid, glutamic acid or serine. When -(A)ₙ- comprises a PEG moiety, it preferably comprises units derived from oligomerisation of the monodisperse PEG-like structures of Formulae IIIA or IIIB:

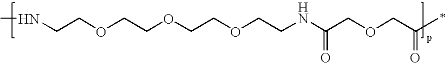

### 17-amino-5-oxo-6-aza-3,9,12,15-tetraoxaheptadecanoic acid of Formula IIIA (IIIA)

wherein p is an integer from 1 to 10 and where the C-terminal unit (*) is connected to the imaging moiety. Alternatively, a PEG-like structure based on a propionic acid derivative of Formula IIIB can be used:

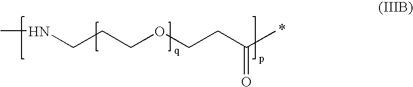

where p is as defined for Formula IIIA and q is an integer from 3 to 15.

In Formula IIIB, p is preferably 1 or 2, and q is preferably 5 to 12.

When the linker group does not comprise PEG or a peptide chain, preferred -(A)ₙ- groups have a backbone chain of linked atoms which make up the -(A)ₙ- moiety of 2 to 10 atoms, most preferably 2 to 5 atoms, with 2 or 3 atoms being especially preferred. A minimum linker group backbone chain of 2 atoms confers the advantage that the imaging moiety is well-separated from the metalloproteinase inhibitor so that any interaction is minimised.

Non-peptide linker groups such as alkylene groups or arylene groups have the advantage that there are no significant hydrogen bonding interactions with the conjugated MMP inhibitor, so that the linker does not wrap round onto the MMP inhibitor. Preferred alkylene spacer groups are —(CH₂)_{d}— where d is 2 to 5. Preferred arylene spacers are of formula:

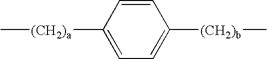

where: a and b are independently 0, 1 or 2.

The linker group -(A)ₙ- preferably comprises a diglycolic acid moiety, a maleimide moiety, a glutaric acid, succinic acid, a polyethyleneglycol based unit or a PEG-like unit of Formula IIIA or IIIB.

When the imaging moiety comprises a metal ion, the metal ion is present as a metal complex. Such metalloproteinase inhibitor conjugates with metal ions are therefore suitably of Formula IIa:

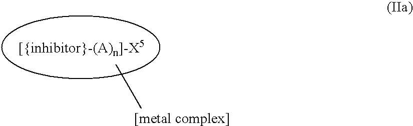

where: A and n are as defined for Formula II above.

By the term “metal complex” is meant a coordination complex of the metal ion with one or more ligands. It is strongly preferred that the metal complex is “resistant to transchelation”, ie. does not readily undergo ligand exchange with other potentially competing ligands for the metal coordination sites. Potentially competing ligands include the hydroxamic acid MMPi moiety itself plus other excipients in the preparation in vitro (eg. radioprotectants or antimicrobial preservatives used in the preparation), or endogenous compounds in vivo (eg. glutathione, transferrin or plasma proteins).

The metal complexes of Formula II are derived from conjugates (ie. conjugated metal-coordinating ligands) of Formula IIb:

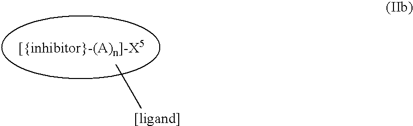

where: A and n are as defined for Formula II above.

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as mibi (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium which form metal complexes resistant to transchelation include, but are not limited to:

(i) diaminedioximes of formula:

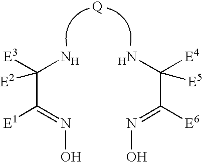

where E¹-E⁶ are each independently an R′ group;
each R′ is H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₀ aminoalkyl, or two or more R′ groups together with the atoms to which they are attached form a carbocyclic, heterocyclic, saturated or unsaturated ring, and wherein one or more of the R′groups is conjugated to the MMP inhibitor;
and Q is a bridging group of formula -(J)_{f}-;
where f is 3, 4 or 5 and each J is independently —O—, —NR′— or —C(R′)₂— provided that -(J)_{f}- contains a maximum of one J group which is —O— or —NR′—.

Preferred Q groups are as follows:

Q=—(CH₂)(CHR′)(CH₂)— ie. propyleneamine oxime or PnAO derivatives;
Q=—(CH₂)₂(CHR′)(CH₂)₂— ie. pentyleneamine oxime or PentAO derivatives;

### Q=—(CH₂)₂NR′(CH₂)₂—.

E¹ to E⁶ are preferably chosen from: C₁₋₃ alkyl, alkylaryl alkoxyalkyl, hydroxyalkyl, fluoroalkyl, carboxyalkyl or aminoalkyl. Most preferably, each E¹ to E⁶ group is CH₃.

The MMP inhibitor is preferably conjugated at either the E¹ or E⁶ R′group, or an R′group of the Q moiety. Most preferably, the MMP inhibitor is conjugated to an R′group of the Q moiety. When the MMP inhibitor is conjugated to an R′group of the Q moiety, the R′group is preferably at the bridgehead position. In that case, Q is preferably —(CH₂)(CHR′)(CH₂)—, —(CH₂)₂(CHR′)(CH₂)₂— or —(CH₂)₂NR′(CH₂)₂—, most preferably —(CH₂)₂(CHR′)(CH₂)₂—. An especially preferred bifunctional diaminedioxime chelator has the Formula:

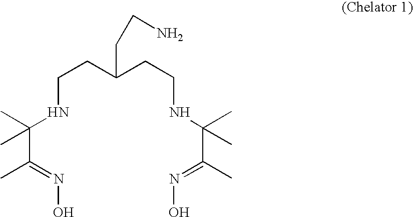

such that the MMP inhibitor is conjugated via the bridgehead —CH₂CH₂NH₂ group.
(ii) N₃S ligands having a thioltriamide donor set such as MAG₃ (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam or dioxocyclam.
(v) N₂O₂ ligands having a diaminediphenol donor set.

The above described ligands are particularly suitable for complexing technetium eg. ^{94m}Tc or ^{99m}Tc, and are described more fully by Jurisson et al [Chem. Rev., 99, 2205-2218 (1999)]. The ligands are also useful for other metals, such as copper (⁶⁴Cu or ⁶⁷Cu), vanadium (eg. ⁴⁸V), iron (eg. ⁵²Fe), or cobalt (eg. ⁵⁵Co). Other suitable ligands are described in Sandoz WO 91/01144, which includes ligands which are particularly suitable for indium, yttrium and gadolinium, especially macrocyclic aminocarboxylate and aminophosphonic acid ligands. Ligands which form non-ionic (i.e. neutral) metal complexes of gadolinium are known and are described in U.S. Pat. No. 4,885,363. When the radiometal ion is technetium, the ligand is preferably a chelating agent which is tetradentate. Preferred chelating agents for technetium are the diaminedioximes, or those having an N₂S₂ or N₃S donor set as described above.

Polydentate hydroxamic acids which are chelating agents are known to form metal complexes with radiometals, including ^{99m}Tc [Safavy et al, Bioconj. Chem., 4, 194-198 (1993)]. The present inventors have, however, found that for monodentate hydroxamic acids [eg. when X¹ is H in Formula (I)], the hydroxamic acid MMPi may compete effectively with the conjugated ligand for the radiometal. Hence, when X¹ is H particular care is needed in the selection of the ligand, ie. it is necessary to choose a ligand which competes effectively with the hydroxamic acid MMPi for the radiometal, to avoid formation of undesirable [hydroxamic acid]-[radiometal] metal complexes. Suitable such ligands include: phosphines; isonitriles; N₄ chelating agents having a tetramine, amidetriamine or diamidediamine donor set; N₃S chelating agents having a thioltriamide donor or diamidepyridinethiol donor set; or N₂S₂ chelating agents having a diaminedithiol donor set such as BAT or an amideaminedithiol donor set such as MAMA. Preferred such ligands include: the N₄, N₃S and N₂S₂ chelating agents described above, most preferably N₄ tetramine and N₂S₂ diaminedithiol or diamidedithiol chelating agents, especially the N₂S₂ diaminedithiol chelator known as BAT:

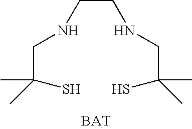

It is strongly preferred that the matrix metalloproteinase inhibitor is bound to the metal complex in such a way that the linkage does not undergo facile metabolism in blood, since that would result in the metal complex being cleaved off before the labelled metalloproteinase inhibitor reached the desired in vivo target site. The matrix metalloproteinase inhibitor is therefore preferably covalently bound to the metal complexes of the present invention via linkages which are not readily metabolised. When the imaging moiety is a radioactive halogen, such as iodine, the MMP inhibitor is suitably chosen to include: a non-radioactive halogen atom such as an aryl iodide or bromide (to permit radioiodine exchange); an activated aryl ring (e.g. a phenol group); an organometallic precursor compound (eg. trialkyltin or trialkylsilyl); an organic precursor such as triazenes or a good leaving group for nucleophilic substitution such as an iodonium salt. Methods of introducing radioactive halogens (including ¹²³I and ¹⁸F) are described by Bolton [J. Lab. Comp. Radiopharm., 45, 485-528 (2002)]. Examples of suitable aryl groups to which radioactive halogens, especially iodine can be attached are given below:

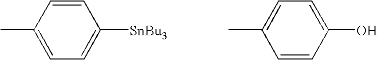

Both contain substituents which permit facile radioiodine substitution onto the aromatic ring. Alternative substituents containing radioactive iodine can be synthesised by direct iodination via radiohalogen exchange, e.g.

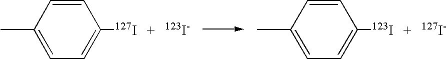

When the imaging moiety is a radioactive isotope of iodine the radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

When the imaging moiety comprises a radioactive isotope of fluorine (eg. ¹⁸F), the radioiodine atom may be carried out via direct labelling using the reaction of ¹⁸F-fluoride with a suitable precursor having a good leaving group, such as an alkyl bromide, alkyl mesylate or alkyl tosylate. ¹⁸F can also be introduced by N-alkylation of amine precursors with alkylating agents such as ¹⁸F(CH₂)₃₀ Ms (where Ms is mesylate) to give N—(CH₂)₃¹⁸F, or O-alkylation of hydroxyl groups with ¹⁸F(CH₂)₃₀ Ms or ¹⁸F(CH₂)₃Br. ¹⁸F can also be introduced by alkylation of N-haloacetyl groups with a ¹⁸F(CH₂)₃OH reactant, to give —NH(CO)CH₂—O—(CH₂)₃¹⁸F derivatives. For aryl systems, ¹⁸F-fluoride nucleophilic displacement from an aryl diazonium salt, aryl nitro compound or an aryl quaternary ammonium salt are possible routes to aryl-¹⁸F derivatives.

Primary amine-containing MMP is of Formula (I) can also be labelled with ¹⁸F by reductive amination, eg:

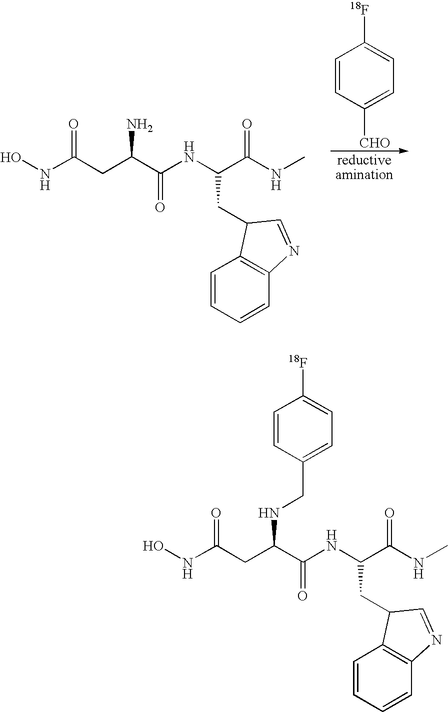

as taught by Kahn et al [J. Lab. Comp. Radiopharm. 45, 1045-1053 (2002)] and Borch et al [J. Am. Chem. Soc. 93, 2897 (1971)]. This approach can also usefully be applied to aryl primary amines, such as compounds comprising phenyl-NH₂ or phenyl-CH₂NH₂ groups.

Amine-containing MMP inhibitors of Formula (I) can also be labelled with ¹⁸F by reaction with ¹⁸F-labelled active esters such as:

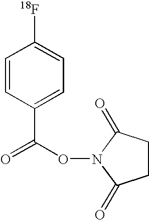

to give amide bond linked products. The N-hydroxysuccinimide ester shown and its use to label peptides is taught by Vaidyanathan et al [Nucl. Med. Biol., 19(3), 275-281 (1992)] and Johnstrom et al [Clin. Sci., 103 (Suppl. 48), 45-85 (2002)]. Further details of synthetic routes to ¹⁸F-labelled derivatives are described by Bolton, J. Lab. Comp. Radiopharm., 45, 485-528 (2002).

Introduction of PET radioisotope labels at the X¹ position can be achieved by eg. O-alkylation of the corresponding hydroxamic acid derivative (X¹═H) with triflate derivatives such as ¹¹CH₃OSO₂CF₃ as taught by Fei et al [J. Lab. Comp. Radiopharm., 46, 343-351 (2003)], or Zheng et al [Nucl. Med. Biol., 30, 753-760 (2003)], or the ¹⁸F O-alkylating reagents described above. ¹¹C PET radiolabels can also be introduced by use of the above triflate derivative to alkylate phenolic hydroxyl groups as taught by Zheng et al [Nucl. Med. Biol., 31, 77-85 (2004)]. Further methods of labelling with “C are taught by Antoni et al [Chapter 5 pages 141-194 in “Handbook of Radiopharmaceuticals”, M. J. Welch and C. S. Redvanly (Eds.), Wiley (2003)].

A preferred class of matrix metalloproteinase inhibitors of the present invention are of Formula IV:

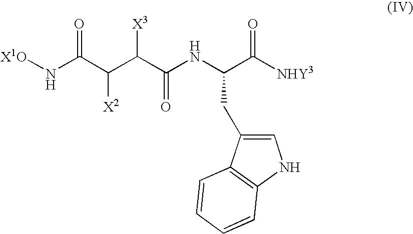

where:
   X¹, X² and X³ are as defined for Formula (I) above;
   Y³ is a Y group as defined in Formula (I) above.

In Formula (IV), Y³ is preferably C₁₋₁₀ alkyl C₁₋₁₀ fluoroalkyl or —(CH₂)_{w}CONHY², most preferably C₁₋₄ alkyl, C₁₋₄ fluoroalkyl or —(CH₂)CONHY², with Y³ equal to —CH₃ or —(CH₂)CONHAr¹ being especially preferred.

Compounds of Formula IV preferably have the stereochemistry corresponding to Formula Ia and Ib (above). Preferred X¹, X² and X³ substituents of Formula (IV) are those described as preferred for Formula (I). X¹ in Formula IV is most preferably H.

When the imaging agent comprises a MMP inhibitor of Formula IV, and the imaging moiety is a gamma-emitting radioactive halogen, the imaging moiety is preferably attached at either the Y³ or X³ substituents, most preferably at the Y³ substituent. When the imaging moiety is a positron-emitting radioactive non-metal, it is preferably attached at the X¹, X³ or Y³, most preferably the Y³ or X³ positions, especially Y³. When X¹ is H, the positron-emitting radioactive non-metal is most preferably attached at the Y³ or X³ positions, most preferably the Y³ position.

When the imaging moiety is a radioactive or paramagnetic metal ion, one of the Y³ or X³ substituents is preferably attached to or comprises the imaging moiety. Most preferably, the Y³ substituent of Formula IV is preferably attached to or comprises the radioactive or paramagnetic metal ion imaging moiety.

A further group of preferred matrix metalloproteinase inhibitors of the present invention are of Formula V:

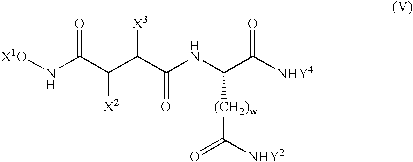

where:
X¹, X² and X³ are as defined for Formula (I) above;
Y⁴ is a Y group as defined in Formula (I) above.

In Formula (V), Y⁴ is preferably C₁₋₁₀ alkyl or Cl₁₋₁₀ fluoroalkyl, most preferably C₁₋₄ alkyl or C₁₋₄ fluoroalkyl, with Y⁴ equal to —CH₃ being especially preferred.

Compounds of Formula V preferably have the stereochemistry corresponding to Formula Ia and Ib (above). Preferred X¹, X² and X³ substituents of Formula (V) are those described as preferred for Formula (I). X¹ in Formula V is most preferably H.

When the imaging agent comprises an MMP inhibitor of Formula V, and the imaging moiety is a gamma-emitting radioactive halogen, the imaging moiety is preferably attached at either the Y², Y⁴ or X³ substituents, most preferably at the Y² or Y⁴ substituents, especially Y². When the imaging moiety is a positron-emitting radioactive non-metal, it is preferably attached at the X¹, X³, Y² or Y⁴ positions, most preferably the Y² or Y⁴ substituents, especially Y². When X¹ is H, the positron-emitting radioactive non-metal is most preferably attached at the Y² or X³ positions, most preferably the Y² position.

When the imaging moiety is a radioactive or paramagnetic metal ion, one of the Y² or Y⁴ substituents is preferably attached to or comprises the imaging moiety. Most preferably, the Y² substituent of Formula V is preferably attached to or comprises the radioactive or paramagnetic metal ion imaging moiety.

When the imaging agent of the present invention comprises a radioactive or paramagnetic metal ion, the metal ion is suitably present as a metal complex. Such metal complexes are suitably prepared by reaction of the conjugate of Formula IIb with the appropriate metal ion. The ligand-conjugate or chelator-conjugate of the MMP inhibitor of Formula IIb can be prepared via the bifunctional chelate approach. Thus, it is well known to prepare ligands or chelating agents which have attached thereto a functional group (“bifunctional linkers” or “bifunctional chelates” respectively). Functional groups that have been attached include: amine, thiocyanate, maleimide and active esters such as N-hydroxysuccinimide or pentafluorophenol. Chelator 1 of the present invention is an example of an amine-functionalised bifunctional chelate. Bifunctional chelates based on thiolactones, which can be used to prepare BAT chelator-conjugates are described by Baidoo et al [Bioconj. Chem., 5, 114-118 (1994)]. Bifunctional chelates suitable for complexation to a technetium or rhenium tricarbonyl core are described by Stichelberger et. al [Nucl. Med. Biol., 30 465-470 (2003)]. Bifunctional HYNIC ligands are described by Edwards et al [Bioconj. Chem., 8, 146 (1997)]. Such bifunctional chelates can be reacted with suitable functional groups on the matrix metalloproteinase inhibitor to form the desired conjugate. Such suitable functional groups on the inhibitor include:

carboxyls (for amide bond formation with an amine-functionalised bifunctional chelator); amines (for amide bond formation with an carboxyl- or active ester-functionalised bifunctional chelator);
halogens, mesylates and tosylates (for N-alkylation of an amine-functionalised bifunctional chelator) and
thiols (for reaction with a maleimide-functionalised bifunctional chelator).

The radiolabelling of the MMP inhibitors of the present invention can be conveniently carried out using “precursors”. When the imaging moiety comprises a metal ion, such precursors suitably comprise “conjugates” of the MMP inhibitor with a ligand, as described in the fourth embodiment below. When the imaging moiety comprises a non-metallic radioisotope, ie. a gamma-emitting radioactive halogen or a positron-emitting radioactive non-metal, such “precursors” suitably comprise a non-radioactive material which is designed so that chemical reaction with a convenient chemical form of the desired non-metallic radioisotope can be conducted in the minimum number of steps (ideally a single step), and without the need for significant purification (ideally no further purification) to give the desired radioactive product. Such precursors can conveniently be obtained in good chemical purity and, optionally supplied in sterile form.

It is envisaged that “precursors” (including ligand conjugates) for radiolabelling of the MMP inhibitors of the present invention can be prepared as follows:

The terminal —OH group of an —N(CH₂)₂OH or —N(CH₂)₃OH derivative may be converted to a tosyl or mesyl group or bromo derivative, which can then be used to conjugate an amino-functionalised chelator. Such tosylate, mesylate or bromo groups of the precursors described may alternatively be displaced with [¹⁸F]fluoride to give an ¹⁸F-labelled PET imaging agent.

Radioiodine derivatives can be prepared from the corresponding phenol precursors. Alkyl bromide derivatives may be used for N-alkylation of an amine-functionalised chelator. Phenyl iodide derivatives can be converted to organometallic precursors for radioiodination compounds, such as trialkyltin or aryl trimethylsilyl (TMS) precursors. Phenyl iodide derivatives can also be converted to an aryl iodonium precursor for radiofluorination with ¹⁵F-fluoride.

Primary amine-functionalised MMP inhibitors may be reacted with acid anhydrides to give N-functionalised precursors of the type —N(CO)(CH₂)₃CO₂H, which can then be conjugated to bifunctional amine-containing ligands. Such primary amine substituted MMP is can be prepared by alkylation of bromo derivatives with benzylamine, followed by removal of the benzyl protecting group under standard conditions such as hydrogenation using a palladium catalyst on charcoal.

Amine-functionalised MMP is may be conjugated directly with a carboxyl- or active ester-functionalised bifunctional chelator, or via a linker. Such compounds may also be reacted with a alkylating agent suitable for ¹⁸F labelling such as ¹⁸F(CH₂)₂OTs (where Ts is a tosylate group) or ¹⁸F(CH₂)₂₀ Ms (where Ms is a mesylate group), to give the corresponding N-functionalised amine derivative having an —N(CH₂)₂¹⁸F substituent. Alternatively, the amine can first be reacted with chloroacetyl chloride to give the —N(CO)CH₂Cl N-derivatised amide, followed by reaction with HS(CH₂)₃¹⁸F or HO(CH₂)₃¹⁸F to give the —N(CO)CH₂S(CH₂)₃¹⁸F and —N(CO)CH₂—O—(CH₂)₃¹⁸F products respectively.

The radiometal complexes of the present invention may be prepared by reaction of a solution of the radiometal in the appropriate oxidation state with the ligand conjugate of Formula IIb at the appropriate pH. The solution may preferably contain a ligand which complexes weakly to the metal (such as gluconate or citrate) i.e. the radiometal complex is prepared by ligand exchange or transchelation. Such conditions are useful to suppress undesirable side reactions such as hydrolysis of the metal ion. When the radiometal ion is ^{99m}Tc, the usual starting material is sodium pertechnetate from a ⁹⁹Mo generator. Technetium is present in ^{99m}Tc-pertechnetate in the Tc(VII) oxidation state, which is relatively unreactive. The preparation of technetium complexes of lower oxidation state Tc(I) to Tc(V) therefore usually requires the addition of a suitable pharmaceutically acceptable reducing agent such as sodium dithionite, sodium bisulphite, ascorbic acid, formamidine sulphinic acid, stannous ion, Fe(II) or Cu(I), to facilitate complexation. The pharmaceutically acceptable reducing agent is preferably a stannous salt, most preferably stannous chloride, stannous fluoride or stannous tartrate.

When the imaging moiety is a hyperpolarised NMR-active nucleus, such as a hyperpolarised ¹³C atom, the desired hyperpolarised compound can be prepared by polarisation exchange from a hyperpolarised gas (such as ¹²⁹Xe or ³He) to a suitable ¹³C-enriched hydroxamic acid derivative.

Some of the metalloproteinase inhibitors of the present invention (eg. Compound 17, Galardin™ Sigma-Aldrich; M5939) are commercially available. Others may be synthesised according to the methods of Levy et al [J. Med. Chem., 41, 199-223 (1998)], and Galardy [Drugs Future, 18, 1109-1111 (1993)]. Further synthetic details are given in Schemes 1 to 4 (below), plus the Examples. When X³ comprises an amino group, the —NHCH(X³)—CO— residue corresponds to an amino acid, which can thus be coupled to the NH₂CH(X⁴)—CO— amino acid residue by conventional solid phase peptide synthesis techniques as described in P. Lloyd-Williams, F. Albericio and E. Girald; Chemical Approaches to the Synthesis of Peptides and Proteins, CRC Press, 1997.

Solid phase peptide synthesis techniques are also expected to provide the useful synthetic disconnections shown in Scheme 5. The steps would be:

(i) Rink amide-Resin (commercially available from Novabiochem) the aminoxy function can be directly incorporated using the commercially available derivative Fmoc-Ams(Boc)-OH (Novabiochem, where Ams is aminoserine), ie. Fmoc(NH)—CH(CO₂H)CH₂O—NH(Boc);
(ii) couple a protected amino acid (AA)—shown as Fmoc-AA-OH, which permits a range of substituents at the R′position (see Scheme 5);
(iii) couple standard L-Tryptophan;
(iv) couple t-butyl protected hydroxamate component;
(v) couple 4-[¹⁸F]fluorobenzaldehyde to the final product.

An alternative in step (i) would be to employ a suitably protected lysine derivative, wherein the epsilon amino group is modified to give the amino acid side chain —(CH₂)₄NH(CO)CH₂O—NH(Boc).

The following abbreviations are used:

Boc=tert-butyloxycarbonyl.

DIC=2-(dimethylamino)isopropyl chloride hydrochloride.

DIEA=Diisopropylethylamine.

DMF═N,N′-dimethylformamide.

HBTU=O-Benzotriazol-1-yl-N,N,N′,N′-tetramethyluronium hexafluorophosphate.

RCP=radiochemical purity.

TES=N-tris(hydroxymethyl)methyl-2-aminoethane sulfonic acid.

TFA=trifluoroacetic acid.

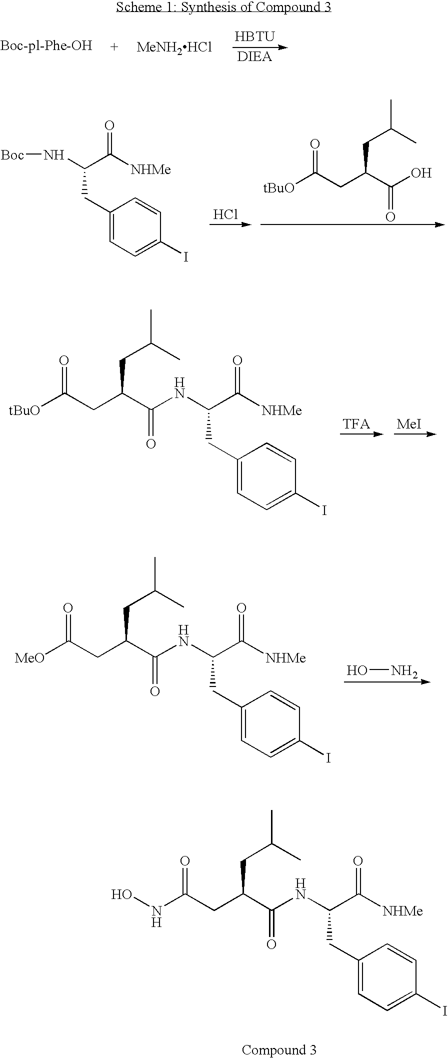

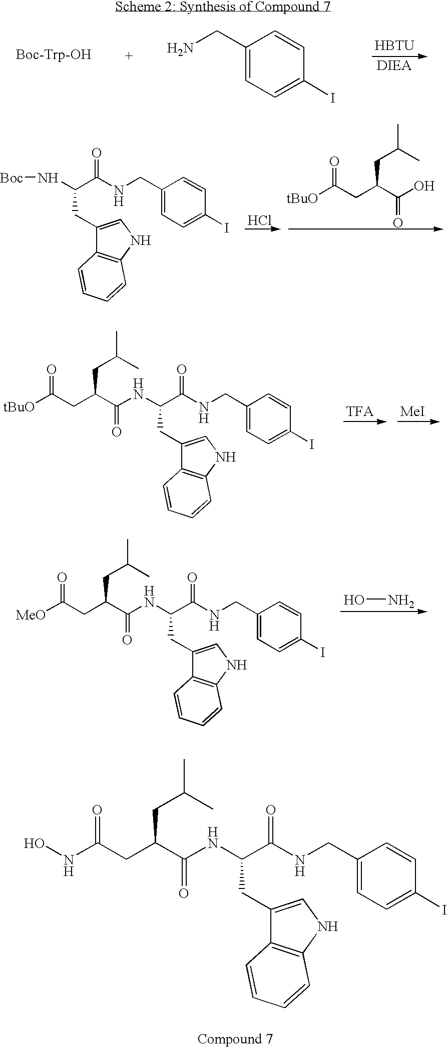

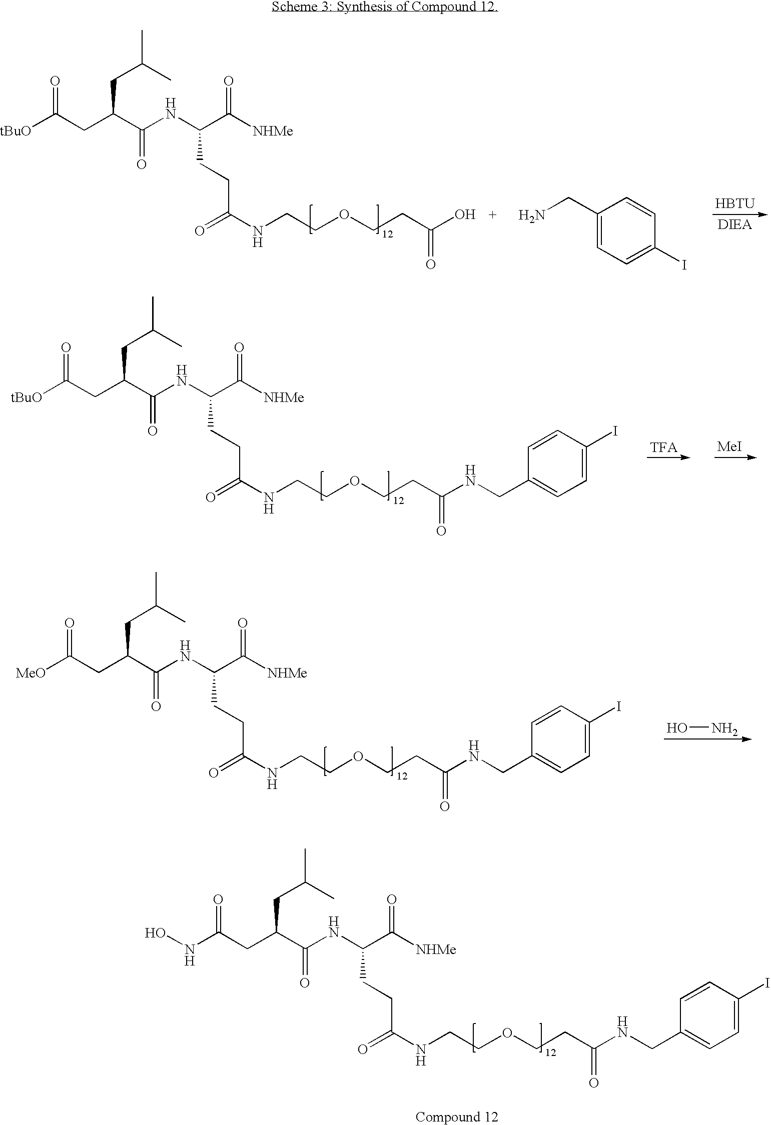

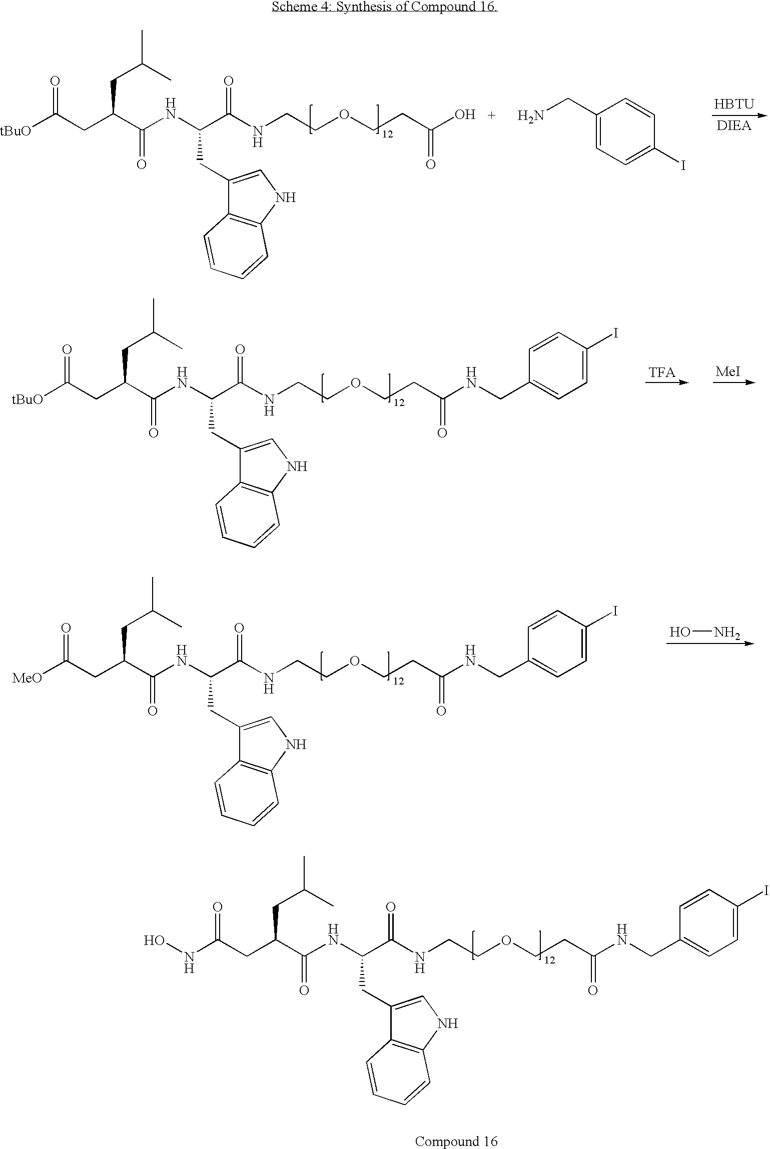

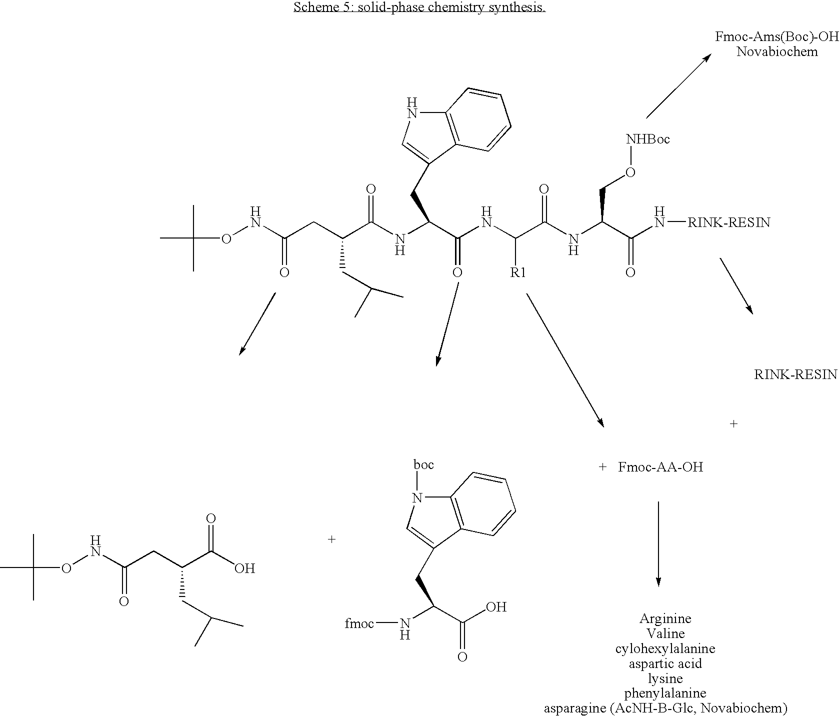

In a second aspect, the present invention provides a pharmaceutical composition which comprises the imaging agent as described above, together with a biocompatible carrier, in a form suitable for mammalian administration. The “biocompatible carrier” is a fluid, especially a liquid, which in which the imaging agent can be suspended or dissolved, such that the composition is physiologically tolerable, ie. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like).

In a third aspect, the present invention provides a radiopharmaceutical composition which comprises the imaging agent as described above wherein the imaging moiety is radioactive, together with a biocompatible carrier (as defined above), in a form suitable for mammalian administration. Such radiopharmaceuticals are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, and are therefore preferably a disposable or other syringe suitable for clinical use. The pre-filled syringe may optionally be provided with a syringe shield to protect the operator from radioactive dose. Suitable such radiopharmaceutical syringe shields are known in the art and preferably comprise either lead or tungsten.

When the imaging moiety comprises ^{99m}Tc, a radioactivity content suitable for a diagnostic imaging radiopharmaceutical is in the range 180 to 1500 MBq of ^{99m}Tc, depending on the site to be imaged in vivo, the uptake and the target to background ratio.

In a fourth aspect, the present invention provides a conjugate of the matrix metalloproteinase inhibitor of Formula (I) with a ligand. Said ligand conjugates are useful for the preparation of matrix metalloproteinase inhibitors labelled with either a radioactive metal ion or a paramagnetic metal ion. Preferably, the ligand conjugate is of Formula IIa, as defined above. Most preferably, the MMP inhibitor of the ligand conjugate is of Formula IV, as defined above. The ligand of the conjugate of the fourth aspect of the invention is preferably a chelating agent. Preferably, the chelating agent has a diaminedioxime, N₂S₂, or N₃S donor set.

In a fifth aspect, the present invention provides precursors useful in the preparation of radiopharmaceutical preparations where the imaging moiety comprises a non-metallic radioisotope, ie. a gamma-emitting radioactive halogen or a positron-emitting radioactive non-metal. Such “precursors” suitably comprise a non-radioactive derivative of the matrix metalloproteinase inhibitor material which is designed so that chemical reaction with a convenient chemical form of the desired non-metallic radioisotope can be conducted in the minimum number of steps (ideally a single step), and without the need for significant purification (ideally no further purification) to give the desired radioactive product. Such precursors can conveniently be obtained in good chemical purity. Suitable precursors are derived from examples described in Bolton, J. Lab. Comp. Radiopharm., 45 485-528 (2002).

Preferred precursors of this embodiment comprise a derivative which either undergoes electrophilic or nucleophilic halogenation; undergoes facile alkylation with an alkylating agent chosen from an alkyl or fluoroalkyl halide, tosylate, triflate (ie. trifluoromethanesulphonate) or mesylate; or alkylates thiol moieties to form thioether linkages. Examples of the first category are:
(a) organometallic derivatives such as a trialkylstannane (eg. trimethylstannyl or tributylstannyl), or a trialkylsilane (eg. trimethylsilyl);
(b) a non-radioactive alkyl iodide or alkyl bromide for halogen exchange and alkyl tosylate, mesylate or triflate for nucleophilic halogenation;
(c) aromatic rings activated towards electrophilic halogenation (eg. phenols) and aromatic rings activated towards nucleophilic halogenation (eg. aryl iodonium, aryl diazonium, nitroaryl).

Preferred derivatives which undergo facile alkylation are alcohols, phenols or amine groups, especially phenols and sterically-unhindered primary or secondary amines.

Preferred derivatives which alkylate thiol-containing radioisotope reactants are N-haloacetyl groups, especially N-chloroacetyl and N-bromoacetyl derivatives.

When X¹ in Formula I is H, suitable precursors for MMPi's of Formula I may therefore comprise a derivative where X¹ is a protecting group (P^{G}) for the hydroxamic acid moiety. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: benzyl, acetyl, benzoyl, trityl (Trt) or trialkylsilyl such as tetrabutyldimethylsilyl. For thiol groups, suitable protecting groups are: trityl and 4-methoxybenzyl. Preferred protecting groups for the hydroxyl group of a hydroxamic acid moiety are: benzyl or trialkylsilyl. The use of further protecting groups are described in ‘Protective Groups in Organic Synthesis’, Theorodora W. Greene and Peter G. M. Wuts, (Third Edition, John Wiley & Sons, 1999).

Preferred convenient chemical forms of the desired non-metallic radioisotope include:
(a) halide ions (eg. ¹²³I-iodide or ¹⁸F-fluoride), especially in aqueous media, for substitution reactions;
(b) ¹¹C-methyl iodide or ¹⁸F-fluoroalkylene compounds having a good leaving group, such as bromide, mesylate or tosylate;
(c) HS(CH₂)₃¹⁸F for S-alkylation reactions with alkylating precursors such as N-chloroacetyl or N-bromoacetyl derivatives.

Examples of suitable such “precursors”, and methods for their preparation are described in the first embodiment (above).

In a sixth aspect, the present invention provides a non-radioactive kit for the preparation of the radiopharmaceutical composition described above where the imaging moiety comprises a radiometal, which comprises a conjugate of a ligand with the matrix metalloproteinase inhibitor of Formula (I). When the radiometal is ^{99m}Tc, the kit suitably further comprises a biocompatible reductant. The ligand conjugates, and preferred aspects thereof, are described in the fourth embodiment above.

Such kits are designed to give sterile radiopharmaceutical products suitable for human administration, e.g. via direct injection into the bloodstream. For ^{99m}Tc, the kit is preferably lyophilised and is designed to be reconstituted with sterile ^{99m}Tc-pertechnetate (TcO₄⁻) from a ^{99m}Tc radioisotope generator to give a solution suitable for human administration without further manipulation. Suitable kits comprise a container (eg. a septum-sealed vial) containing the ligand or chelator conjugate in either free base or acid salt form, together with a biocompatible reductant such as sodium dithionite, sodium bisulphite, ascorbic acid, formamidine sulphinic acid, stannous ion, Fe(II) or Cu(I). The biocompatible reductant is preferably a stannous salt such as stannous chloride or stannous tartrate. Alternatively, the kit may optionally contain a metal complex which, upon addition of the radiometal, undergoes transmetallation (i.e. metal exchange) giving the desired product.

The non-radioactive kits may optionally further comprise additional components such as a transchelator, radioprotectant, antimicrobial preservative, pH-adjusting agent or filler. The “transchelator” is a compound which reacts rapidly to form a weak complex with technetium, then is displaced by the ligand. This minimises the risk of formation of reduced hydrolysed technetium (RHT) due to rapid reduction of pertechnetate competing with technetium complexation. Suitable such transchelators are salts of a weak organic acid, ie. an organic acid having a pKa in the range 3 to 7, with a biocompatible cation. Suitable such weak organic acids are acetic acid, citric acid, tartaric acid, gluconic acid, glucoheptonic acid, benzoic acid, phenols or phosphonic acids. Hence, suitable salts are acetates, citrates, tartrates, gluconates, glucoheptonates, benzoates, phenolates or phosphonates. Preferred such salts are tartrates, gluconates, glucoheptonates, benzoates, or phosphonates, most preferably phosphonates, most especially diphosphonates. A preferred such transchelator is a salt of MDP, ie. methylenediphosphonic acid, with a biocompatible cation.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (ie. 4-aminobenzoic acid), gentisic acid (ie. 2,5-dihydroxybenzoic acid) and salts thereof with a biocompatible cation as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition post-reconstitution, ie. in the radioactive diagnostic product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

In a seventh aspect, the present invention provides kits for the preparation of radiopharmaceutical preparations where the imaging moiety comprises a non-metallic radioisotope, ie. a gamma-emitting radioactive halogen or a positron-emitting radioactive non-metal. Such kits comprise the “precursor” of the fifth embodiment, preferably in sterile non-pyrogenic form, so that reaction with a sterile source of the radioisotope gives the desired radiopharmaceutical with the minimum number of manipulations. Such considerations are particularly important for radiopharmaceuticals where the radioisotope has a relatively short half-life, and for ease of handling and hence reduced radiation dose for the radiopharmacist. Hence, the reaction medium for reconstitution of such kits is preferably aqueous, and in a form suitable for mammalian administration.

The “precursor” of the kit is preferably supplied covalently attached to a solid support matrix. In that way, the desired radiopharmaceutical product forms in solution, whereas starting materials and impurities remain bound to the solid phase. Precursors for solid phase electrophilic fluorination with ¹⁸F-fluoride are described in WO 03/002489. Precursors for solid phase nucleophilic fluorination with ¹⁸F-fluoride are described in WO 03/002157. The kit may therefore contain a cartridge which can be plugged into a suitably adapted automated synthesizer. The cartridge may contain, apart from the solid support-bound precursor, a column to remove unwanted fluoride ion, and an appropriate vessel connected so as to allow the reaction mixture to be evaporated and allow the product to be formulated as required. The reagents and solvents and other consumables required for the synthesis may also be included together with a compact disc carrying the software which allows the synthesiser to be operated in a way so as to meet the customer requirements for radioactive concentration, volumes, time of delivery etc. Conveniently, all components of the kit are disposable to minimise the possibility of contamination between runs and will be sterile and quality assured.

In an eighth aspect, the present invention discloses the use of the matrix metalloproteinase inhibitor imaging agent described above for the diagnostic imaging of atherosclerosis, especially unstable vulnerable plaques.

In a further aspect, the present invention discloses the use of the matrix metalloproteinase inhibitor imaging agent described above for the diagnostic imaging of other inflammatory diseases, cancer, or degenerative diseases.

In a further aspect, the present invention discloses the use of the matrix metalloproteinase inhibitor imaging agent described above for the intravascular detection of atherosclerosis, especially unstable vulnerable plaques, using proximity detection. Such proximity detection may be achieved using intravascular devices such as catheters or intra-operatively using hand-held detectors (eg. gamma detectors). Such intravascular detection is particularly useful when the imaging moiety is a reporter group suitable for in vivo optical imaging or a β-emitter, since such moieties may not be readily detected outside the mammalian body, but are suitable for proximity detection.

The invention is illustrated by the non-limiting Examples detailed below. Example 1 provides the synthesis of two iodine-containing MMPi derivatives (Compounds 2 and 3). Examples 2, 4, 5 and 7 give the synthesis of various tributyltin precursors useful for radiohalogenation, especially radioiodination. Example 3 provides the synthesis of two indolyl compounds of Formula IV (Compounds 6 and 7). Example 6 provides the synthesis of a derivative with a linker group attached at the X⁴ position. Example 8 provides the synthesis of a derivative with a linker group attached at the Y¹ position. Example 9 describes the synthesis of the compound 1,1,1-tris(2-aminoethyl)methane. Example 10 provides an alternative synthesis of 1,1,1-tris(2-aminoethyl)methane which avoids the use of potentially hazardous azide intermediates. Example 11 describes the synthesis of a chloronitrosoalkane precursor. Example 12 describes the synthesis of a preferred amine-substituted bifunctional diaminedioxime of the present invention (Chelator 1). Example 13 provides the synthesis of an ¹⁸F derivative suitable for N-alkylation. Example 14 provides the synthesis of an ¹⁸F thiol derivative suitable for S-alkylation. Example 15 provides a method of radioiodination of trialkyltin precursors with the radioisotope ¹²³I. Example 16 provides a general method of radiolabelling MMPi-chelator conjugates with the radioisotope ^{99m}Tc.

Example 17 provides in vitro MMPi inhibition assays, plus MMP-1, MMP-2, MMP-9 and MMP-12 potency results for several compounds of the invention. The results confirm high potency [in the nM to sub-nM range] for a range of MMP inhibitors. Such “broad-spectrum” potency is of particular advantage for targeting some diseases such as vulnerable plaques in atherosclerosis because several MMPs are up-regulated in the disease process. The ability of the MMPi described herein to target these MMPs (particularly collagenases and gelatinases) leads to maximal accumulation of the MMPi at the site of pathology.

Example 18 provides animal biodistribution data for a representative ¹²³I-labelled compound of the invention (Compound 2A) in an in vivo lesion (Lewis Lung Carcinoma or LLC) which is know to express active MMPs. Compound 2A exhibited tumour uptake and retention between 5 and 120 minutes post injection, consistent with specific retention in the MMP-expression tumour tissue. In contrast, clearance from normal tissues (e.g. blood and other background tissues) occurred between 5 and 120 minutes p.i., supporting a targeting mechanism which is specific for MMP-expressing tumour.

Example 19 provides animal biodistribution data for Compounds 2A, 6A and 18A in the ApoE ligated animal model of MMP expression. Compound 2A exhibited carotid uptake and retention between 5 and 120 minutes post injection, consistent with specific retention in the MMP-rich lesion tissue. In contrast, clearance from normal tissues [e.g. blood and other background tissues] was significant, with good carotid to blood ratios, indicating a targeting mechanism which is specific for MMP-expressing lesion tissue. Examples 20 to 24 provide the syntheses of Compounds 9, 10, 13, 14 and 18-21.

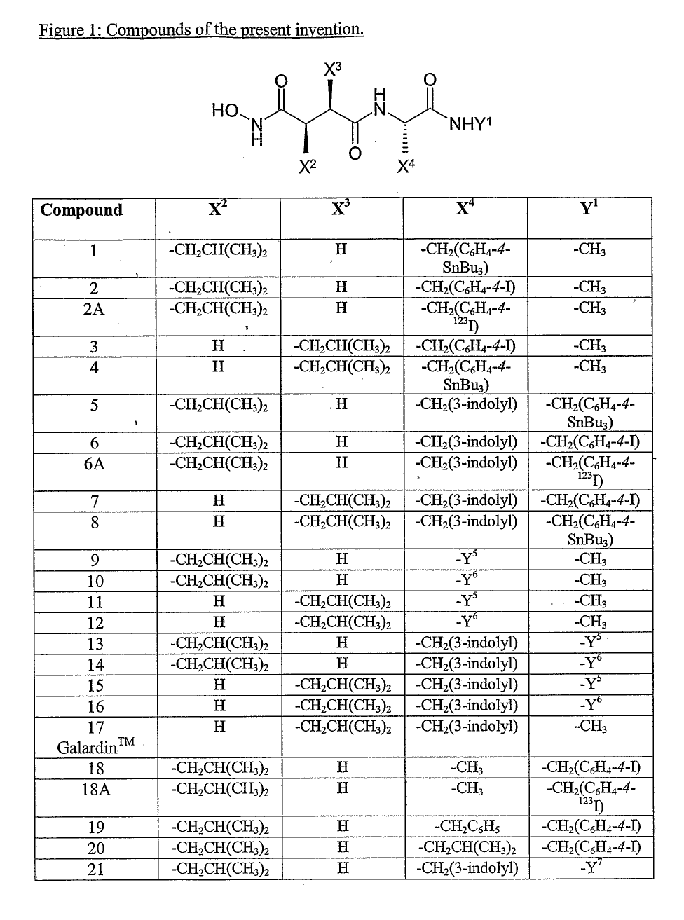

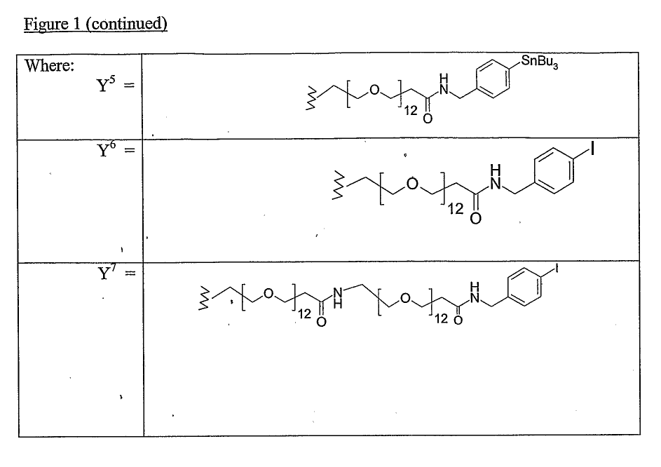

FIG. 1 shows the chemical structures of several compounds of the invention.

### EXAMPLE 1

### Synthesis of Compounds 2 and 3

Compound 3 was prepared according to Scheme 1. Coupling of Boc-pI-Phe-OH with MeNH₂.HCl in the presence of DIEA using HBTU as coupling reagent afforded the fully protected phenylalanine. Removal of the Boc group by acidolysis (HCl in dioxane) followed by coupling with (R)-2-isobutylsuccinic acid-4-t-butyl ester (see Example 3) gave the intermediate shown. Following cleavage of the t-butyl group under acidic conditions (TFA/TES/CH₂Cl₂), the carboxylic acid was converted to the methyl ester utilizing iodomethane. The methyl ester was treated with hydroxylamine under basic conditions (partial racemisation was observed) to give a solid (crude yield 54.1%). The crude product was purified by RP-HPLC using TFA/water/acetonitrile as solvent. The pure fractions were collected and freeze-dried to afford a white solid (global yield 27.7%). HPLC analysis 93%

Compound 2 was prepared in an analogous manner. Crude yield 38.3% Global yield 16.4% HPLC analysis 95%

### EXAMPLE 2

### Synthesis of Tributyltin Precursor Compounds 1 and 4

Compound 3 (purified) was used as starting material, and the reaction performed under a nitrogen atmosphere. Compound 3 was treated with bis(tributyltin) using Pd(PPh₃)₄ as catalyst. The reaction mixture was heated under reflux in a mixture toluene/acetonitrile (3/25). The crude product was isolated as a solid (crude yield 57.5%). The crude Compound 4 product was purified by RP-HPLC using AcO⁻N₄⁺/H₂O/acetonitrile as solvent (global yield 4.2%). HPLC analysis 90.2%

Compound 1 was prepared in an analogous manner from Compound 2. Crude yield 65.5% Global yield 11.2% HPLC analysis 98.8%

### EXAMPLE 3

### Synthesis of Compounds 6 and 7

Compound 7 was prepared according to Scheme 1. Coupling of Boc-Trp-OH with 4-iodobenzylamine in the presence of DIEA using HBTU as coupling reagent afforded the fully protected Tryptophan. Removal of the Boc group by acidolysis (HCl in dioxane) followed by coupling with (R)-2-isobutylsuccinic acid-4-t-butyl ester [prepared according to the method of Levy, D. F. et al. (1998) J. Med. Chem., 41, 199-223] gave the intermediate shown. Following cleavage of the t-butyl group under acidic conditions (TFA/TES/CH₂Cl₂), the carboxylic acid was converted to the methyl ester using iodomethane. The methyl ester was treated with hydroxylamine under basic conditions to give a solid (crude yield 62.8%). The crude product was purified by RP-HPLC using TFA/H₂O/acetonitrile as solvent. The pure fractions were collected and freeze-dried to afford a white solid (global yield 21.6%). HPLC analysis 93.3%

Compound 6 was prepared in an analogous manner. Crude yield 70.4% Global yield 44.9% HPLC analysis 95%

### EXAMPLE 4

### Synthesis of Trialkyltin Precursor Compounds 5 and 8

Compound 7 (crude) was treated with bis(tributyltin) using Pd(PPh₃)₄ as catalyst under a nitrogen atmosphere, in a manner similar to Example 21. The reaction mixture was heated under reflux in a mixture of toluene/acetonitrile (3/25). The crude product was isolated as a solid (crude yield 59.1%). The crude Compound 8 product was purified by RP-HPLC using AcO⁻N₄⁺/H₂O/acetonitrile as solvent (global yield 2%). HPLC analysis 84.7%

Compound 5 was prepared an analogous manner from Compound 6 (crude). Some degradation of the product was observed during the reaction. Attempted RP-HPLC purification was ineffective due to solubility (soluble in DMSO and insoluble in acetonitrile and methanol). Crude yield 68% Global yield 12.5% HPLC analysis 57.4%

### EXAMPLE 5

### Synthesis of Compound 11

Purified Compound 12 was treated with bis(tributyltin) using Pd(PPh₃)₄ as catalyst under a nitrogen atmosphere. The reaction mixture was heated under reflux in a mixture of toluene/acetonitrile (3/25). The crude product was isolated as an oil (crude yield 100%). The crude product was purified by RP-HPLC with AcO⁻H₄⁺/H₂O/Acetonitrile as solvent (global yield 16.6%). HPLC analysis 45.6%* *This compound was obtained as an oil and underwent some degradation during freeze-drying.

### EXAMPLE 6

### Synthesis of Compound 12

This compound was synthesised by coupling in solution using a protected fragment, which was prepared using solid phase synthesis.

### Step(a): Solid Phase Synthesis of Protected Peptide Fragment.

The coupling of the amino acids was performed step by step on chlorotrityl PS resin (0.8 meq/g). Fmoc-PEG-OH was coupled to Chlorotrityl PS resin in DMF in the presence of DIEA. The deprotection/coupling cycle was described below:

2 eq of Fmoc-Amino acid and 2 eq of HOBt were dissolved in DMF (2-3 ml per mmole of amino acid). The solution was poured into the reaction vessel containing the resin. 2 eq of DIC were added.

| Step | Solvent | Time | Cycle |
|---|---|---|---|
| 1 | Coupling/DMF | (*) min | Coupling |
| 2 | DMF | 3 × 1 min | Washing |
| 3 | Piperidine/DMF (25%) | 1 min (*) | Deprotection |
| 4 | Piperidine/DMF (25%) | 2 × 15 min | Deprotection |
| 5 | DMF | 7 × 1 min (*) | Washing |

(*) Completion of coupling was determined by the Kaiser test. [E. Kaiser et al. Anal. Biochem. 34, 595 (1970)].

The cleavage of the peptide from the resin was performed using 1% TFA in CH₂Cl₂. The crude product was obtained as an oil. Crude yield 60.7%

### Step(b): Synthesis in Solution.

The product from Step (a) was coupled with 4-iodobenzylamine in the presence of DIEA using HBTU as coupling reagent. Following removal of the t-butyl group under acidic conditions (TFA/TES/CH₂Cl₂), the carboxylic acid was converted to the methyl ester using iodomethane. The methyl ester was treated with hydroxylamine under basic conditions. The crude product was obtained as an oil (crude yield 72.5%). The crude product was purified by RP-HPLC using TFA/H₂O/acetonitrile as solvent. The pure fractions were collected and freeze-dried to give an oil (global yield 18.5%). HPLC analysis 98.3%

### EXAMPLE 7

### Synthesis of Compound 15

Purified Compound 16 was treated with bis(tributyltin) using Pd(PPh₃)₄ as catalyst under a nitrogen atmosphere. The reaction mixture was heated under reflux in a mixture of toluene/acetonitrile (3/25). The crude product was isolated as an oil (crude yield 100%). The crude product was purified by RP-HPLC with AcO⁻NH₄⁺/H₂O/Acetonitrile as solvent to afford an oil (global yield 14.4%). HPLC analysis 91.3% The tributyltin precursor to Compound 18A was prepared from Compound 18 in a similar manner. Purity by HPLC=93.3% ESI-MS: m/z=714.5 [M−H]

### EXAMPLE 8

### Synthesis of Compound 16

Compound 16 was prepared according to Scheme 4. Compound 16 was synthesised by coupling in solution using a protected peptide fragment prepared via solid phase synthesis.

### Step (a): Solid Phase Synthesis of Protected Peptide Fragment.

The coupling of the amino acids was performed step by step on chlorotrityl PS resin (0.8 meq/g). Fmoc-PEG-OH was coupled to chlorotrityl PS resin in DMF in the presence of DIEA. The deprotection/coupling cycle is described below:

2 eq of Fmoc-Amino acid and 2 eq of HOBt were dissolved in DMF (2-3 ml per mmole of amino acid). The solution was poured into the reaction vessel containing the resin. 2 eq of DIC were added.

| Step | Solvent | Time | Cycle |
|---|---|---|---|
| 1 | Coupling/DMF | (*) min | Coupling |
| 2 | DMF | 3 × 1 min | Washing |
| 3 | Piperidine/DMF (25%) | 1 min (*) | Deprotection |
| 4 | Piperidine/DMF (25%) | 2 × 15 min | Deprotection |
| 5 | DMF | 7 × 1 min (*) | Washing |

(*) Completion of coupling was determined by the Kaiser test (see Example 6).

The cleavage of the peptide from the resin was performed using 1% TFA in CH₂Cl₂. The crude product was obtained as an oil. Crude yield 100%

### Step (b): Synthesis in Solution.

The protected peptide form Step (a) was coupled with 4-iodobenzylamine in the presence of DIEA using HBTU as coupling reagent afforded compound 12. Following removal of the t-butyl group under acidic conditions (TFA/TES/CH₂Cl₂), the carboxylic acid was converted to the methyl ester utilising iodomethane. The methyl ester was treated with hydroxylamine under basic conditions. The crude product was obtained as an oil (crude yield 43.1%). The crude product was purified by RP-HPLC using TFA/H₂O/acetonitrile as solvent. The pure fractions were collected and freeze-dried to give Compound 16 as an oil (global yield 6%). HPLC analysis 87.7%

### EXAMPLE 9

### Synthesis of 1,1,1-tris(2-aminoethyl)methane

(Step a): 3-(methoxycarbonylmethylene)glutaric acid dimethylester.

Carbomethoxymethylenetriphenylphosphorane (167 g, 0.5 mol) in toluene (600 ml) was treated with dimethyl 3-oxoglutarate (87 g, 0.5 mol) and the reaction heated to 100° C. on an oil bath at 120° C. under an atmosphere of nitrogen for 36 h. The reaction was then concentrated in vacuo and the oily residue triturated with 40/60 petrol ether/diethylether 1:1, 600 ml. Triphenylphosphine oxide precipitated out and the supernatant liquid was decanted/filtered off. The residue on evaporation in vacuo was Kugelrohr distilled under high vacuum Bpt (oven temperature 180-200° C. at 0.2 torr) to give 3-(methoxycarbonylmethylene)glutaric acid dimethylester (89.08 g, 53%).

NMR ¹H(CDCl₃): δ 3.31 (2H, s, CH₂), 3.7 (9H, s, 3×OCH₃), 3.87 (2H, s, CH₂), 5.79 (1H, s, ═CH,) ppm.

NMR¹³C(CDCl₃), δ 36.56,CH₃, 48.7, 2×CH₃, 52.09 and 52.5 (2×CH₂); 122.3 and 146.16 C═CH; 165.9, 170.0 and 170.5 3×COO ppm.

(Step b): Hydrogenation of 3-(methoxycarbonylmethylene)glutaric acid dimethylester.

3-(methoxycarbonylmethylene)glutaric acid dimethylester (89 g, 267 mmol) in methanol (200 ml) was shaken with (10% palladium on charcoal: 50% water) (9 g) under an atmosphere of hydrogen gas (3.5 bar) for (30 h). The solution was filtered through kieselguhr and concentrated in vacuo to give 3-(methoxycarbonylmethyl)glutaric acid dimethylester as an oil, yield (84.9 g, 94%).

NMR ¹H(CDCl₃), δ 2.48 (6H, d, J=8 Hz, 3×CH₂), 2.78 (1H, hextet, J=8 Hz CH,) 3.7 (9H, s, 3×CH₃).

NMR¹³C(CDCl₃), δ 28.6, CH; 37.50, 3×CH₃; 51.6, 3×CH₂; 172.28, 3×COO.

### (Step c): Reduction and Esterification of Trimethyl Ester to the Triacetate.

Under an atmosphere of nitrogen in a 3 necked 2 L round bottomed flask lithium aluminium hydride (20 g, 588 mmol) in tetrahydrofuran (400 ml) was treated cautiously with tris(methyloxycarbonylmethyl)methane (40 g, 212 mmol) in tetrahydrofuran (200 ml) over 1 h. A strongly exothermic reaction occurred, causing the solvent to reflux strongly. The reaction was heated on an oil bath at 90° C. at reflux for 3 days. The reaction was quenched by the cautious dropwise addition of acetic acid (100 ml) until the evolution of hydrogen ceased. The stirred reaction mixture was cautiously treated with acetic anhydride solution (500 ml) at such a rate as to cause gentle reflux. The flask was equipped for distillation and stirred and then heating at 90° C. (oil bath temperature) to distil out the tetrahydrofuran. A further portion of acetic anhydride (300 ml) was added, the reaction returned to reflux configuration and stirred and heated in an oil bath at 140° C. for 5 h. The reaction was allowed to cool and filtered. The aluminium oxide precipitate was washed with ethyl acetate and the combined filtrates concentrated on a rotary evaporator at a water bath temperature of 50° C. in vacuo (5 mmHg) to afford an oil. The oil was taken up in ethyl acetate (500 ml) and washed with saturated aqueous potassium carbonate solution. The ethyl acetate solution was separated, dried over sodium sulphate, and concentrated in vacuo to afford an oil. The oil was Kugelrohr distilled in high vacuum to give tris(2-acetoxyethyl)methane (45.3 g, 96%) as an oil. Bp. 220° C. at 0.1 mmHg.

NMR ¹H(CDCl₃), δ 1.66(7H, m, 3×CH₂, CH), 2.08 (1H, s, 3×CH₃); 4.1 (6H, t, 3×CH₂O).

NMR ¹³C(CDCl₃), δ 20.9, CH₃; 29.34, CH; 32.17, CH₂; 62.15, CH₂O; 171, CO.

(Step d): Removal of Acetate Groups from the Triacetate.

Tris(2-acetoxyethyl)methane (45.3 g, 165 mM) in methanol (200 ml) and 880 ammonia (100 ml) was heated on an oil bath at 80° C. for 2 days. The reaction was treated with a further portion of 880 ammonia (50 ml) and heated at 80° C. in an oil bath for 24 h. A further portion of 880 ammonia (50 ml) was added and the reaction heated at 80° C. for 24 h. The reaction was then concentrated in vacuo to remove all solvents to give an oil. This was taken up into 880 ammonia (150 ml) and heated at 80° C. for 24 h. The reaction was then concentrated in vacuo to remove all solvents to give an oil. Kugelrohr distillation gave acetamide bp 170-180 0.2 mm. The bulbs containing the acetamide were washed clean and the distillation continued. Tris(2-hydroxyethyl)methane (22.53 g, 92%) distilled at bp 220° C. 0.2 mm.

NMR ¹H(CDCl₃), δ 1.45(6H, q, 3×CH₂), 2.2 (1H, quintet, CH); 3.7 (6H, t 3×CH₂OH); 5.5 (3H, brs, 3×OH).

NMR¹³C(CDCl₃), δ 22.13, CH; 33.95, 3×CH₂; 57.8, 3×CH₂OH.

(Step e): Conversion of the triol to the tris(methanesulphonate).

To an stirred ice-cooled solution of tris(2-hydroxyethyl)methane (10 g, 0.0676 mol) in dichloromethane (50 ml) was slowly dripped a solution of methanesulphonyl chloride (40 g, 0.349 mol) in dichloromethane (50 ml) under nitrogen at such a rate that the temperature did not rise above 15° C. Pyridine (21.4 g, 0.27 mol, 4 eq) dissolved in dichloromethane (50 ml) was then added drop-wise at such a rate that the temperature did not rise above 15° C., exothermic reaction. The reaction was left to stir at room temperature for 24 h and then treated with 5N hydrochloric acid solution (80 ml) and the layers separated. The aqueous layer was extracted with further dichloromethane (50 ml) and the organic extracts combined, dried over sodium sulphate, filtered and concentrated in vacuo to give tris[2-(methylsulphonyloxy)ethyl]methane contaminated with excess methanesulphonyl chloride. The theoretical yield was 25.8 g.

NMR ¹H(CDCl₃), δ 4.3 (6H, t, 2×CH₂), 3.0 (9H, s, 3×CH₃), 2 (1H, hextet, CH), 1.85 (6H, q, 3×CH₂).

(Step f): Preparation of 1,1,1-tris(2-azidoethyl)methane.

A stirred solution of tris[2-(methylsulphonyloxy)ethyl]methane [from Step 1(e), contaminated with excess methylsulphonyl chloride] (25.8 g, 67 mmol, theoretical) in dry DMF (250 ml) under nitrogen was treated with sodium azide (30.7 g, 0.47 mol) portion-wise over 15 minutes. An exotherm was observed and the reaction was cooled on an ice bath. After 30 minutes, the reaction mixture was heated on an oil bath at 50° C. for 24 h. The reaction became brown in colour. The reaction was allowed to cool, treated with dilute potassium carbonate solution (200 ml) and extracted three times with 40/60 petrol ether/diethylether 10:1 (3×150 ml). The organic extracts were washed with water (2×150 ml), dried over sodium sulphate and filtered. Ethanol (200 ml) was added to the petrol/ether solution to keep the triazide in solution and the volume reduced in vacuo to no less than 200 ml. Ethanol (200 ml) was added and reconcentrated in vacuo to remove the last traces of petrol leaving no less than 200 ml of ethanolic solution. The ethanol solution of triazide was used directly in Step 1(g).

### CARE: DO NOT REMOVE ALL THE SOLVENT AS THE AZIDE IS POTENTIALLY EXPLOSIVE AND SHOULD BE KEPT IN DILUTE SOLUTION AT ALL TIMES.

Less than 0.2 ml of the solution was evaporated in vacuum to remove the ethanol and an NMR run on this small sample:

NMR ¹H(CDCl₃), δ 3.35 (6H, t, 3×CH₂), 1.8 (1H, septet, CH₁), 1.6 (6H, q, 3×CH₂).

(Step g: Preparation of 1,1,1-tris(2-aminoethyl)methane.

Tris(2-azidoethyl)methane (15.06 g, 0.0676 mol), (assuming 100% yield from previous reaction) in ethanol (200 ml) was treated with 10% palladium on charcoal (2 g, 50% water) and hydrogenated for 12 h. The reaction vessel was evacuated every 2 hours to remove nitrogen evolved from the reaction and refilled with hydrogen. A sample was taken for NMR analysis to confirm complete conversion of the triazide to the triamine.

Caution: unreduced azide could explode on distillation. The reaction was filtered through a Celite pad to remove the catalyst and concentrated in vacuo to give tris(2-aminoethyl)methane as an oil. This was further purified by Kugelrohr distillation bp. 180-200° C. at 0.4 mm/Hg to give a colourless oil (8.1 g, 82.7% overall yield from the triol).

NMR ¹H(CDCl₃), 2.72 (6H, t, 3×CH₂N), 1.41 (H, septet, CH), 1.39 (6H, q, 3×CH₂).

NMR¹³C(CDCl₃), δ 39.8 (CH₂NH₂), 38.2 (CH₂.), 31.0 (CH).

### EXAMPLE 10

### Alternative Preparation of 1,1,1-tris(2-aminoethyl)methane

(Step a): Amidation of trimethylester with p-methoxy-benzylamine.

Tris(methyloxycarbonylmethyl)methane [2 g, 8.4 mmol; prepared as in Step 1(b) above] was dissolved in p-methoxy-benzylamine (25 g, 178.6 mmol). The apparatus was set up for distillation and heated to 120° C. for 24 hrs under nitrogen flow. The progress of the reaction was monitored by the amount of methanol collected. The reaction mixture was cooled to ambient temperature and 30 ml of ethyl acetate was added, then the precipitated triamide product stirred for 30 min. The triamide was isolated by filtration and the filter cake washed several times with sufficient amounts of ethyl acetate to remove excess p-methoxy-benzylamine. After drying 4.6 g, 100%, of a white powder was obtained. The highly insoluble product was used directly in the next step without further purification or characterisation.

(Step b): Preparation of 1,1,1-tris[2-(p-methoxybenzylamino)ethyl]methane.

To a 1000 ml 3-necked round bottomed flask cooled in a ice-water bath the triamide from step 2(a) (10 g, 17.89 mmol) is carefully added to 250 ml of 1M borane solution (3.5 g, 244.3 mmol) borane. After complete addition the ice-water bath is removed and the reaction mixture slowly heated to 60° C. The reaction mixture is stirred at 60° C. for 20 hrs. A sample of the reaction mixture (1 ml) was withdrawn, and mixed with 0.5 ml 5N HCl and left standing for 30 min. To the sample 0.5 ml of 50 NaOH was added, followed by 2 ml of water and the solution was stirred until all of the white precipitate dissolved. The solution was extracted with ether (5 ml) and evaporated. The residue was dissolved in acetonitrile at a concentration of 1 mg/ml and analysed by MS. If mono- and diamide (M+H/z=520 and 534) are seen in the MS spectrum, the reaction is not complete. To complete the reaction, a further 100 ml of 1M borane THF solution is added and the reaction mixture stirred for 6 more hrs at 60° C. and a new sample withdrawn following the previous sampling procedure. Further addition of the 1M borane in THF solution is continued as necessary until there is complete conversion to the triamine.

The reaction mixture is cooled to ambient temperature and 5N HCl is slowly added, [CARE: vigorous foam formation occurs!]. HCl was added until no more gas evolution is observed. The mixture was stirred for 30 min and then evaporated. The cake was suspended in aqueous NaOH solution (20-40%; 1:2 w/v) and stirred for 30 minutes. The mixture was then diluted with water (3 volumes). The mixture was then extracted with diethylether (2×150 ml) [CARE: do not use halogenated solvents]. The combined organic phases were then washed with water (1×200 ml), brine (150 ml) and dried over magnesium sulphate. Yield after evaporation: 7.6 g, 84% as oil.

NMR ¹H(CDCl₃), δ: 1.45, (6H, m, 3×CH₂; 1.54, (1H, septet, CH); 2.60 (6H, t, 3×CH₂N); 3.68 (6H, s, ArCH₂); 3.78 (9H, s, 3×CH₃O); 6.94 (6H, d, 6×Ar). 7.20(6H, d, 6×Ar).

NMR¹³C(CDCl₃), δ: 32.17,CH; 34.44, CH₂; 47.00, CH₂; 53.56, ArCH₂; 55.25, CH₃O; 113.78, Ar; 129.29, Ar; 132.61; Ar; 158.60, Ar;

(Step c): Preparation of 1,1,1-tris(2-aminoethyl)methane.

1,1,1-tris[2-(p-methoxybenzylamino)ethyl]methane (20.0 gram, 0.036 mol) was dissolved in methanol (100 ml) and Pd(OH)₂ (5.0 gram) was added. The mixture was hydrogenated (3 bar, 100° C., in an autoclave) and stirred for 5 hours. Pd(OH)₂ was added in two more portions (2×5 gram) after 10 and 15 hours respectively. The reaction mixture was filtered and the filtrate was washed with methanol. The combined organic phase was evaporated and the residue was distilled under vacuum (1×10⁻², 110° C.) to give 2.60 gram (50%) of 1,1,1-tris(2-aminoethyl)methane identical with the previously described Example 1.

### EXAMPLE 11

### Preparation of 3-chloro-3-methyl-2-nitrosobutane

A mixture of 2-methylbut-2-ene (147 ml, 1.4 mol) and isoamyl nitrite (156 ml, 1.16 mol) was cooled to −30° C. in a bath of cardice and methanol and vigorously stirred with an overhead air stirrer and treated dropwise with concentrated hydrochloric acid (140 ml, 1.68 mol) at such a rate that the temperature was maintained below −20° C. This requires about 1 h as there is a significant exotherm and care must be taken to prevent overheating. Ethanol (100 ml) was added to reduce the viscosity of the slurry that had formed at the end of the addition and the reaction stirred at −20 to −10° C. for a further 2 h to complete the reaction. The precipitate was collected by filtration under vacuum and washed with 4×30 ml of cold (−20° C.) ethanol and 100 ml of ice cold water, and dried in vacuo to give 3-chloro-3-methyl-2-nitrosobutane as a white solid. The ethanol filtrate and washings were combined and diluted with water (200 ml) and cooled and allowed to stand for 1 h at −10° C. when a further crop of 3-chloro-3-methyl-2-nitrosobutane crystallised out. The precipitate was collected by filtration and washed with the minimum of water and dried in vacuo to give a total yield of 3-chloro-3-methyl-2-nitrosobutane (115 g 0.85 mol, 73%)>98% pure by NMR.

NMR ¹H(CDCl₃), As a mixture of isomers (isomer1, 90%) 1.5 d, (2H, CH₃), 1.65 d, (4H, 2×CH₃), 5.85,q, and 5.95,q, together 1H (isomer2, 10%), 1.76 s, (6H, 2×CH₃), 2.07 (3H, CH₃).

### EXAMPLE 12

### Synthesis of bis[N-(1,1-dimethyl-2-N-hydroxyimine propyl)-2-aminoethyl]-(2-aminoethyl)methane (Chelator 1)

To a solution of tris(2-aminoethyl)methane (4.047 g, 27.9 mmol) in dry ethanol (30 ml) was added potassium carbonate anhydrous (7.7 g, 55.8 mmol, 2 eq) at room temperature with vigorous stirring under a nitrogen atmosphere. A solution of 3-chloro-3-methyl-2-nitrosobutane (7.56 g, 55.8 mol, 2 eq) was dissolved in dry ethanol (100 ml) and 75 ml of this solution was dripped slowly into the reaction mixture. The reaction was followed by TLC on silica [plates run in dichloromethane, methanol, concentrated (0.88 sg) ammonia; 100/30/5 and the TLC plate developed by spraying with ninhydrin and heating]. The mono-, di- and tri-alkylated products were seen with RF's increasing in that order. Analytical HPLC was run using RPR reverse phase column in a gradient of 7.5-75% acetonitrile in 3% aqueous ammonia. The reaction was concentrated in vacuo to remove the ethanol and resuspended in water (110 ml). The aqueous slurry was extracted with ether (100 ml) to remove some of the trialkylated compound and lipophilic impurities leaving the mono and desired dialkylated product in the water layer. The aqueous solution was buffered with ammonium acetate (2 eq, 4.3 g, 55.8 mmol) to ensure good chromatography. The aqueous solution was stored at 4° C. overnight before purifying by automated preparative HPLC.

Yield (2.2 g, 6.4 mmol, 23%).

Mass spec; Positive ion 10 V cone voltage. Found: 344; calculated M+H=344.

NMR ¹H(CDCl₃), δ 1.24(6H, s, 2×CH₃), 1.3 (6H, s, 2×CH₃), 1.25-1.75 (7H, m, 3×CH₂,CH), (3H, s, 2×CH₂), 2.58 (4H, m, CH₂N), 2.88 (2H, t CH₂N₂), 5.0 (6H, s, NH₂, 2×NH, 2×OH).

NMR ¹H((CD₃)₂SO) δ 1.1 4×CH; 1.29, 3×CH₂; 2.1 (4H, t, 2×CH₂);

NMR ¹³C((CD₃)₂SO), δ 9.0 (4×CH₃), 25.8 (2×CH₃), 31.0 2×CH₂, 34.6 CH₂, 56.8 2×CH₂N; 160.3; C═N.

HPLC conditions: flow rate 8 m1 min using a 25 mm PRP column A=3% ammonia solution (sp.gr=0.88)/water; B=Acetonitrile

| Time | % B |
|---|---|
| 0 | 7.5 |
| 15 | 75.0 |
| 20 | 75.0 |
| 22 | 7.5 |
| 30 | 7.5 |

Load 3 ml of aqueous solution per run, and collect in a time window of 12.5-13.5 min.

### EXAMPLE 13

### Synthesis of the ¹⁸F-Labelled Derivative for N-alkylation

Synthesis of 3-[¹⁸F] fluoropropyl tosylate

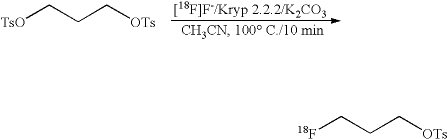

Via a two-way tap Kryptofix 222 (10 mg) in acetonitrile (300 μl) and potassium carbonate (4 mg) in water (300 μl), prepared in a glass vial, was transferred using a plastic syringe (1 ml) into a carbon glass reaction vessel sited in a brass heater. ¹⁸F-fluoride (185-370 MBq) in the target water (0.5-2 ml) was then added through the two-way tap. The heater was set at 125° C. and the timer started. After 15 mins three aliquots of acetonitrile (0.5 ml) were added at 1 min intervals. The ¹⁸F-fluoride was dried up to 40 mins in total. After 40 mins, the heater was cooled down with compressed air, the pot lid was removed and 1,3-propanediol-di-p-tosylate (5-12 mg) and acetonitrile (1 ml) was added. The pot lid was replaced and the lines capped off with stoppers. The heater was set at 100° C. and labelled at 100° C./10 mins. After labelling, 3-[18F] fluoropropyl tosylate was isolated by Gilson RP HPLC using the following conditions:

| | |
|---|---|
| Column | u-bondapak C18 7.8 × 300 mm |
| Eluent | Water (pump A): Acetonitrile (pump B) |
| Loop Size | 1 ml |
| Pump speed | 4 ml/min |
| Wavelength | 254 nm |
| Gradient | 5-90% eluent B over 20 min |
| Product Rt | 12 min |

Once isolated, the cut sample (ca. 10 ml) was diluted with water (10 ml) and loaded onto a conditioned C18 sep pak. The sep pak was dried with nitrogen for 15 mins and flushed off with an organic solvent, pyridine (2 ml), acetonitrile (2 ml) or DMF (2 ml). Approx. 99% of the activity was flushed off.

3-[¹⁸F] fluoropropyl tosylate is used to N-alkylate amines by refluxing in pyridine.

### EXAMPLE 14

### [¹⁸F]-Thiol Derivative for S-alkylation

Step (a): Preparation of 3-[¹⁸F] fluoro-tritylsulfanyl-propane.

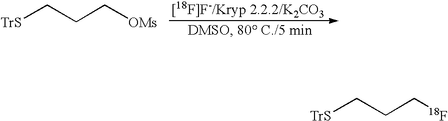

Via a two-way tap Kryptofix 222 (10 mg) in acetonitrile (800 μl) and potassium carbonate (1 mg) in water (50 μl), prepared in a glass vial, was transferred using a plastic syringe (1 ml) to the carbon glass reaction vessel situated in the brass heater. ¹⁸F-fluoride (185-370 MBq) in the target water (0.5-2 ml) was then also added through the two-way tap. The heater was set at 125° C. and the timer started. After 15 mins three aliquots of acetonitrile (0.5 ml) were added at 1 min intervals. The ¹⁸F-fluoride was dried up to 40 mins in total. After 40 mins, the heater was cooled down with compressed air, the pot lid was removed and trimethyl-(3-tritylsulfanyl-propoxy)silane (1-2 mg) and DMSO (0.2 ml) was added. The pot lid was replaced and the lines capped off with stoppers. The heater was set at 80° C. and labelled at 80° C./5 mins. After labelling, the reaction mixture was analysed by RP HPLC using the following HPLC conditions:

| | |
|---|---|
| Column | u-bondapak C18 7.8 × 300 mm |
| Eluent | 0.1% TFA/Water (pump A): 0.1% TFA/ |
| | Acetonitrile (pump B) |
| Loop Size | 100 ul |
| Pump speed | 4 ml/min |
| Wavelength | 254 nm |
| Gradient | 1 mins 40% B |
| | 15 mins 40-80% B |
| | 5 mins 80% B |

The reaction mixture was diluted with DMSO/water (1:1 v/v, 0.15 ml) and loaded onto a conditioned t-C18 sep-pak. The cartridge was washed with water (10 ml), dried with nitrogen and 3-[¹⁸F] fluoro-1-tritylsulfanyl-propane was eluted with 4 aliquots of acetonitrile (0.5 ml per aliquot).

Step (b): Preparation of 3-[¹⁸F]fluoro-propane-1-thiol

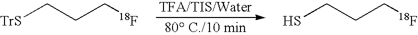

A solution of 3-[¹⁸F] fluoro-1-tritylsulfanyl-propane in acetonitrile (1-2 ml) was evaporated to dryness using a stream of nitrogen at 100° C./10 mins. A mixture of TFA (0.05 ml), triisopropylsilane (0.01 ml) and water (0.01 ml) was added followed by heating at 80° C./10 mins to produce 3-[¹⁸F] fluoro-propane-1-thiol.

Step (c): Reaction with —N(CO)CH₂Cl Precursors.

A general procedure for labelling a chloroacetyl precursor is to cool the reaction vessel containing the 3-[¹⁸F] fluoro-1-mercapto-propane from Step (b) with compressed air, and then to add ammonia (27% in water, 0.1 ml) and the precursor (1 mg) in water (0.05 ml). The mixture is heated at 80° C./10 mins.

### EXAMPLE 15

### ¹²³I Radiolabelling of Tributyltin Precursors

The following generally applicable method was used:

10 μl of 1 mM Na¹²⁷I in 0.01 M NaOH was added to 200 μl 0.2 M NH₄OAc (pH 4). The Na¹²⁷I/NH₄OAc solution was then added to 25.0 μl Na¹²³I in 0.05 M NaOH (˜500 MBq; Amersham Cygne). The combined solution was transferred to a silanised plastic vial containing a small glass conical insert. The plastic vial had been silanised using SIGMACOTE™ (chlorinated organopolysiloxane in heptane; Sigma Chemicals). A solution of peracetic acid was prepared by adding 10 μl of 36-40 wt % peracetic acid solution in acetic acid to 5 ml H₂O. 100 μl of the diluted peracetic acid solution was then added to 900 μl H₂O and 10 μl of this dilution was then added to the vial containing the Na¹²³/¹²⁷I. Finally, 64 μl of a 1.5 mM solution of the tributyltin precursor (Compound 1) in a silanised plastic vial was added to the reaction mixture and the solution was allowed to stand for 3 min.

¹²³I-Compound 2 was purified using gradient HPLC chromatography with γ- and UV-detectors and a reverse-phase Phenomenex C₁₈(2) Luna 5μ, 150×4.6 mm column.

| | |
|---|---|
| HPLC-conditions | eluant A: 0.1% TFA in H₂O |
| | eluant B: 0.1% TFA in CH₃CN |
| | eluent B from 30% to 70% over 12 min. |
| | 13 min 100% B |
| | 25 min 100% B |
| | 25.5 min 30% B |
| Flow: | 1 ml/min |
| λ: | 254 nm. |

Thus, 260 μl of the reaction mixture was injected into the HPLC and the peak corresponding to ¹²³I-Compound 2 (7.3 min retention time) was purified into 200 μg of 4-aminobenzoic acid in 200 μl MeOH. The RCP was 47% by HPLC. After removal of the organic solvents in vacuo, the volume was made up to 1.6 ml with 50 mM phosphate buffer (pH 7.4). The final solution containing 73.75 MBq/ml had a pH of 7-7.5. (specific activity=48 MBq/mnole). After leaving at room temperature for 198 min, HPLC showed the RCP of the purified ¹²³I-Compound 2 to be 94%.

The ¹²³I product formed in the reaction (R_{T}=7.3 min) co-elutes with a cold standard of ¹²⁷I-Compound 2 by HPLC. The reaction was also repeated as described above but this time in the absence of Na¹²³I in 0.05 M NaOH. The reaction mixture was analysed by LCMS using electrospray mass spectrometry in the positive ion mode. HPLC conditions were the same as described above but this time using 0.01% TFA in H₂O as eluant A and 0.01% TFA in CH₃CN as eluant B. The product had the same retention time as for authentic non-radioactive Compound 2. Mass spectroscopy of the peak at 5.85 min from the reaction mixture gave a main peak with mass 480.75 (100%).

### EXAMPLE 16

### ^{99m}Tc-Radiolabelling

### General Method

^{99m}Tc complexes may be prepared by adding the following to a nitrogen-purged P46 vial:
1 ml N₂ purged MeOH,
100 μg of the ligand-MMPi conjugate in 100 μl MeOH,
0.5 ml Na₂CO₃/NaHCO₃ buffer (pH 9.2),
0.5 ml TcO₄⁻ from Tc generator,
0.1 ml SnCl₂/MDP solution,
(solution containing 10.2 mg SnCl₂ and 101 mg methylenediphosphonic acid in 100 ml N₂ purged saline).

ITLC (Instant thin layer chromatography) is used to determine the RCP. SG plates and a mobile phase of MeOH/(NH₄OAc 0.1M) 1:1 show RHT (reduced hydrolysed Tc) at the origin, pertechnetate at the solvent front and technetium complexes at an intermediate Rf. The reaction mixture can also be analysed by reverse-phase HPLC (Xterra column RP18 3.5 μm, 100 mm×4.6 mm) using 0.07% ammonia as eluant A and acetonitrile as eluant B.

### EXAMPLE 17

### In Vitro Metalloproteinase Inhibition Assay

Compounds were screened using the following commercially available Biomol assay kits:

MMP-2 colorimetric assay kit-Catalogue number AK-408,
MMP-9 colorimetric assay kit-Catalogue number AK-410,
MMP-12 colorimetric assay kit-Catalogue number AK-402,
Which are available from Affiniti Research Products Ltd. (Palatine House, Matford Court, Exeter, EX2 8NL, UK).

### (a) Test Compound Preparation.

Inhibitors were provided in powdered form, and stored at 4° C. For each inhibitor a 1 mM stock solution in DMSO was prepared, dispensed into 20 μl aliquots and these aliquots stored at −20° C. The stock solution was diluted to give 8 inhibitor concentrations (recommended: 50 μM, 5 μM, 500 mM, 50 mM, 5 nM, 500 μM, 50 μM and 5 μM). Dilutions were made in the kit assay buffer. A five-fold dilution of the inhibitor stocks is made on addition to the assay wells, therefore final concentration range was from 10 μM to 1 pM.

### (b) Experimental Procedure.

Details are provided with the commercial kit, but can be summarised as follows:
Prepare test compound dilutions as above,
Add assay buffer to plate,
Add test compounds to plate
Prepare standard kit inhibitor NNGH (see kit for dilution factor)
Add NNGH to control inhibitor wells
Prepare MMP enzyme (see kit for dilution factor)
Add MMP to plate
Incubate plate at 37° C. for ˜15 min
Prepare thiopeptolide substrate (see kit for dilution factor)
Add substrate to plate
Count every 2 min for 1 hr, 37° C., 414 nm on a Labsystems iEMS plate reader.

### (c) Results.

The results are given in Table 1:

TABLE 1
MMP inhibitor potency data.

| | | | MMP-8 | MMP-9 | MMP-12 |
|---|---|---|---|---|---|
| Compound | MMP-1 [Kᵢ] | MMP-2 (Ki) | (Ki) | (Ki) | (Ki) |
| 2 | 8.83 ± 0.72 nM | 0.42 ± 0.27 nM | 0.57 | 0.23 ± 0.23 nM | 4.18 ± 0.43 nM |
| 3 | 1.77 nM | 0.20 ± 0.11 nM | — | 0.11 ± 0.08 nM | 0.4 ± 0.04 nM |
| 6 | 0.48 ± 0.55 nM | 0.26 ± 0.08 nM | 1.127 ± 1.54 | 0.13 ± 0.14 nM | 0.14 nM |
| 7 | 1.57 ± 2.37 | — | — | — | — |
| 10 | 23.9 | 8.8 ± 3.1 nM | — | 2.85 ± 3.5 nM | 4.7 nM |
| 12 | 28.5 nM | 21 ± 2.2 nM | — | 7.15 ± 3.32 nM | 8.3 nM |
| 14 | 2.68 ± 0.19 nM | 0.21 ± 0.05 nM | 0.082 nM | 0.04 ± 0.03 nM | 0.51 nM |
| 16 | 18 ± 1.69 nM | 0.61 ± 0.16 nM | — | 0.50 ± 0.420 nM | 0.40 ± 0.09 nM |
| 17 | — | 0.69 ± 0.25 nM | — | 1.52 ± 1.79 nM | — |

(Galardin ™)

### EXAMPLE 18

### Biodistribution of Radioiodinated Derivative Compound 2A in an Animal Tumour Model of MMP Expression

The in vivo Lewis Lung [LLC] Carcinoma Tumour model has been used for screening of MMP is due to reproducible up-regulation of several MMPs in the tumour. As such, this model provides a good assessment of the efficacy of the MMP is for in vivo targeting of lesions that express MMPs. Literature reports have shown that LLC cells express pro and active MMP-2 and pro NMP-9 and LLC tumours MMP-2 and MMP-9 (not classified as pro or active) [Bae et al Drugs Exp Clin Res. 29(1):15-23 (2003)].

### Results.

A summary of the results is given in Table 2:

TABLE 2
Biodistribution of Compound 2A in LLC
Tumour Model (15 days tumour growth).

Time Post Injection (Minutes)

| | 5 | 30 | 60 | 120 |
|---|---|---|---|---|
| % ID/g Tumour | 1.39 | 1.08 | 1.21 | 1.35 |
| % ID/g Blood | 5.89 | 2.19 | 1.49 | 1.38 |
| % ID Heart | 0.86 | 0.57 | 0.56 | 0.49 |
| % ID Lung | 1.38 | 0.73 | 0.59 | 0.45 |
| % ID Liver | 29.76 | 24.43 | 20.04 | 13.45 |
| % ID Urinary Excretion | 19.85 | 22.52 | 27.7 | 31.03 |
| % ID GI Excretion | 17.81 | 28.46 | 29.5 | 34.42 |
| % Retained Tumour | — | 78 | 87 | 97 |

### EXAMPLE 19

### Biodistribution of Radioiodinated Derivatives Compounds 2A, 6A and 18A in an ApoE Ligated Animal Model of MMP Expression

The ApoE ligation model was also studied. ApoE mice are transgenic knock-out mice, which lack the ApoE gene, and are therefore unable to regulate their plasma cholesterol levels. As a consequence ApoE mice develop atherosclerotic lesions, a process which is accelerated with feeding of high fat diet. Further acceleration of lesion development can be achieved by ligating the carotid artery, resulting in advanced lesion formation within 4 weeks of surgery and high fat diet feeding. This model has been shown to have levels of tissue remodelling, with high macrophage and MMP expression, and is described by Ivan et al [Circulation. 105, 2686-2691 (2002)]. A summary of the results is given in Table 3:

TABLE 3
Biodistribution of Compounds 2A, 6A
and 18A in the ApoE Ligation Model.

| | Time Post Injection (Minutes) | | |
|---|---|---|---|
| Compound 2A | 5 | 120 | 360 |
| % ID/g Blood | 6.5 | 1.5 | 1.6 |
| % ID/g Heart | 5.5 | 3.6 | 1.5 |
| % ID/g Carotid | 3.3 | 2.7 | 1.6 |
| % Retained Carotid | — | 83 | 47 |
| Carotid: Blood | 0.5 | 1.8 | 1.0 |
| Carotid: Heart | 0.8 | 0.8 | 1.0 |

| Compound 6A | 5 | 60 | 120 |
|---|---|---|---|
| % ID/g Blood | 9.9 | 3.2 | 2.5 |
| % ID/g Heart | 9.8 | 2.2 | 1.5 |
| % ID/g Carotid | 3.9 | 1.4 | 1.4 |
| % Retained Carotid | — | 36 | 37 |
| Carotid: Blood | 0.3 | 0.6 | 0.6 |
| Carotid: Heart | 0.4 | 0.7 | 1.0 |

| Compound 18A | 5 | 120 | 360 |
|---|---|---|---|
| % ID/g Blood | 7.3 | 2.4 | 1.2 |
| % ID/g Heart | 10.1 | 2.6 | 1.0 |
| % ID/g Carotid | 2.9 | 1.0 | 0.8 |
| % Retained Carotid | — | 34 | 26 |
| Carotid: Blood | 0.5 | 0.4 | 0.7 |
| Carotid: Heart | 0.3 | 0.4 | 0.8 |

### EXAMPLE 20

### Synthesis of Compounds 14 and 21

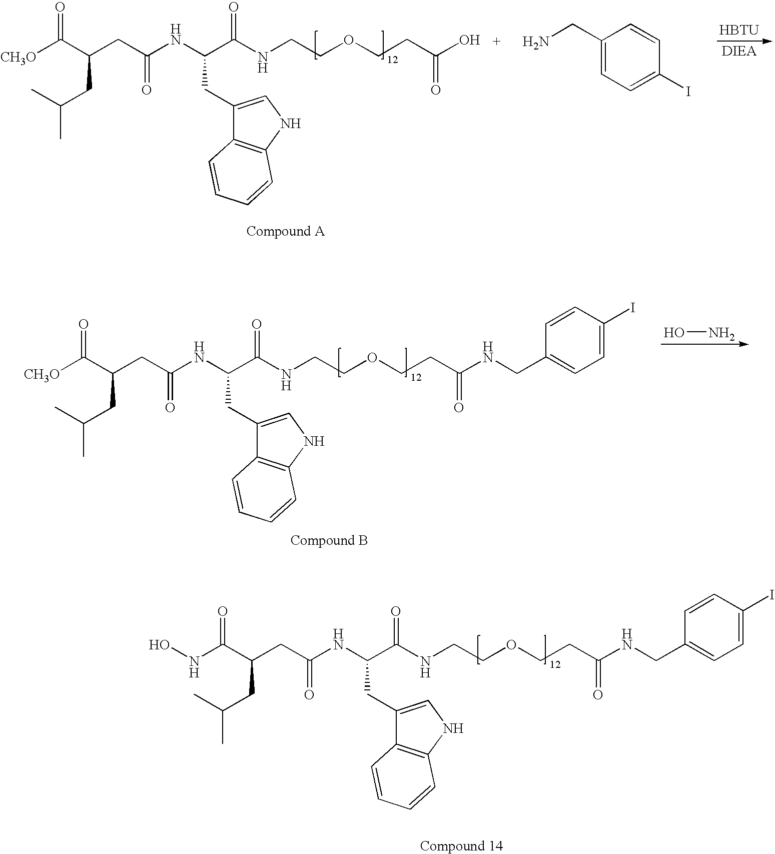

This compound was synthesised by coupling in solution using a protected fragment Compound A. Compound A was prepared using solid phase synthesis. The coupling of the amino acids was performed step by step on chlorotrityl PS resin (0.8 meq/g).

### Step (a): Synthesis of Compound A.

Fmoc-PEG-OH was coupled to Chlorotrityl PS resin in DMF in the presence of DIEA. The deprotection/coupling cycle is described below:

2 eq of Fmoc-Amino acid and 2 eq of HOBt were dissolved in DMF (2-3 ml per mmole of amino acid). The solution was poured into the reaction vessel containing the resin. 2 eq of DIC were added.

| Step | Solvent | Time | Cycle |
|---|---|---|---|
| 1 | Coupling/DMF | (*) min | Coupling |
| 2 | DMF | 3 × 1 min | Washing |
| 3 | Piperidine/DMF (25%) | 1 min (*) | Deprotection |
| 4 | Piperidine/DMF (25%) | 2 × 15 min | Deprotection |
| 5 | DMF | 7 × 1 min (*) | Washing |

(*) Completion of coupling was determined by the Kaiser test.
The cleavage of the peptide from the resin was performed using 1% TFA in CH₂Cl₂.
The crude product was obtained as an oil. Crude yield 38.1%

### Step (b): Synthesis in Solution.

Coupling of Compound A with 4-iodobenzylamine in the presence of DIEA using HBTU as coupling reagent afforded Compound B. Compound B was treated with hydroxylamine under basic conditions. The crude product was obtained as an oil (crude yield 91.6%). The crude product was purified by RP-HPLC using TFA/H₂O/acetonitrile as solvent. The pure fractions were collected and freeze-dried to give an oil (global yield 28.3%). HPLC analysis 90%

Compound 21 was prepared in an analogous manner:

### Purity by HPLC=90%

ESI-MS: m/z=1789.6 [MH]⁺

### EXAMPLE 21

### Synthesis of Compound 13

Purified Compound 14 was used as starting material. The reaction was performed under a nitrogen atmosphere. Compound 14 was treated with bis(tributyltin)(1.5 eq) using Pd(PPh₃)₄(0.05 eq) as catalyst. The reaction mixture was heated under reflux in a mixture of Toluene/Acetonitrile (3/25). The crude product was isolated as an oil (crude yield 100%). The crude product was purified by RP-HPLC with AcO^{-NH}₄⁺/H₂O/Acetonitrile as solvent to afford an oil (global yield 6.85%). HPLC analysis 44%. This compound was obtained as an oil, which underwent degradation during attempted lyophilisation. HPLC analysis before dry-freeze was 90.2%

### EXAMPLE 22

### Synthesis of Compound 10

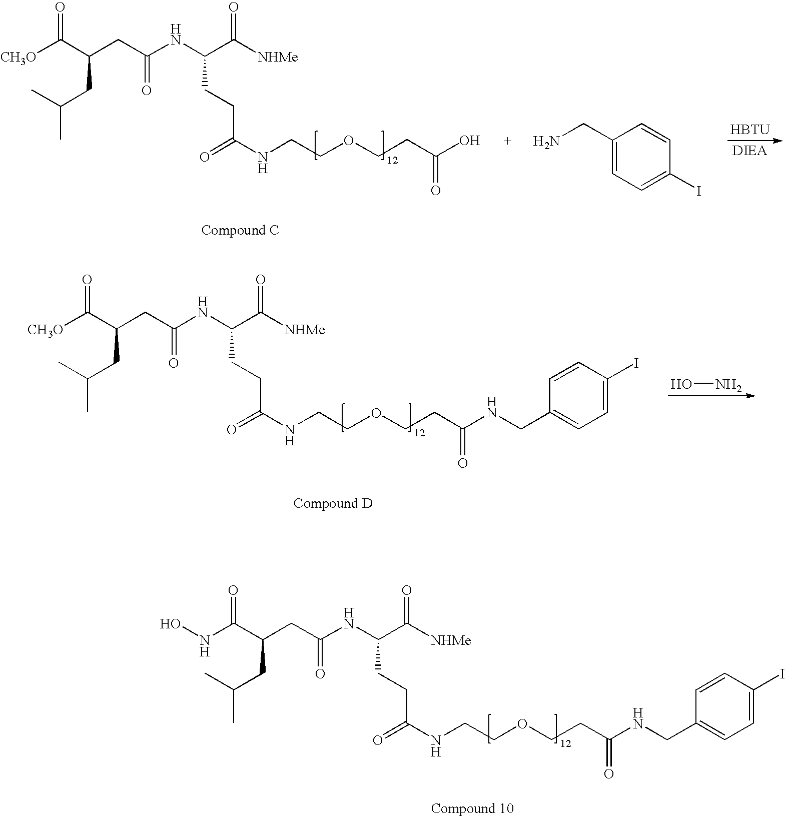

This compound was synthesised by coupling in solution using a protected fragment Compound C. Compound C was prepared using solid phase synthesis. The coupling of the amino acids was performed step by step on chlorotrityl PS resin (0.8 meq/g).

### Step (a): Synthesis of Compound C.

Fmoc-PEG-OH was coupled to Chlorotrityl PS resin in DMF in the presence of DIEA. The deprotection/coupling cycle was described below

2 eq of Fmoc-Amino acid and 2 eq of HOBt were dissolved in DMF (2-3 ml per mmole of amino acid). The solution was poured into the reaction vessel containing the resin. 2 eq of DIC were added.

| Step | Solvent | Time | Cycle |
|---|---|---|---|
| 1 | Coupling/DMF | (*) min | Coupling |
| 2 | DMF | 3 × 1 min | Washing |
| 3 | Piperidine/DMF (25%) | 1 min (*) | Deprotection |
| 4 | Piperidine/DMF (25%) | 2 × 15 min | Deprotection |
| 5 | DMF | 7 × 1 min (*) | Washing |

(*) Completion of coupling was determined by the Kaiser test (see Example 6).

The cleavage of the peptide from the resin was performed using 1% de TFA in DCM. The crude product was obtained as an oil. Crude yield 40.7%.

### Step (b): Synthesis in Solution.

Coupling of Compound C with 4-Iodobenzylamine in the presence of DIEA using HBTU as coupling reagent afforded Compound D. Compound D was treated with hydroxylamine under basic conditions. The crude product was obtained as an oil (crude yield 93.1%). The crude Compound 10 was purified by RP-HPLC using TFA/H₂O/acetonitrile as solvent. The pure fractions were collected and freeze-dried to give an oil (global yield 25%). HPLC analysis 87.2%.

### EXAMPLE 23

### Synthesis of Compound 9

Purified Compound 10 was used as starting material. The reaction was performed under nitrogen atmosphere. Compound 10 was treated with bis(tributyltin)(2×1.5 eq) using Pd(PPh₃)₄(3×0.05 eq) as catalyst. The reaction mixture was heated under reflux in a mixture of Toluene/Acetonitrile (3/25). The crude product was isolated as an oil (crude yield 100%). The crude product was purified by RP-HPLC with AcO⁻NH₄⁺/H₂O/Acetonitrile as solvent (global yield 15%). HPLC analysis 78.8%.

### EXAMPLE 24

### Synthesis of Compounds 18 to 20

Compound 19 was prepared by coupling of Boc-Phe-OH with p-I-benzylamine.HCl in the presence of DIEA using HBTU as coupling reagent to give the fully protected Phenylalanine. Removal of the Boc group by acidolysis (HCl in dioxane) followed by coupling with (R)-2-isobutylsuccinic acid-1-t-butyl ester gave succinate-Phe-p-I -benzylamide fragment. Following cleavage of the t-butyl group under acidic conditions (TFA/TES/DCM), the carboxylic acid was converted to the methyl ester utilizing Iodomethane. The methyl ester was treated with hydroxylamine under basic conditions to give a solid. The crude product was purified by RP-HPLC using TFA/water/acetonitrile as eluent. The pure fractions were collected and freeze dried to afford a white solid.

Purity by HPLC=90.1% ESI-MS: m/z=551.9 [MH]⁺

Compounds 18 and 20 were prepared in an analogous manner:

Compound 18: Purity by HPLC=91% ESI-MS: m/z=475.9 [MH]⁺

Compound 20: Purity by HPLC=96.1% ESI-MS: m/z=516.3 [MH]⁺

1. An imaging agent which comprises a metalloproteinase inhibitor of Formula (I) labelled with an imaging moiety at position X¹, X², X³, X⁴ or Y¹, wherein the imaging moiety can be detected following administration of said labelled matrix metalloproteinase inhibitor to the mammalian body in vivo 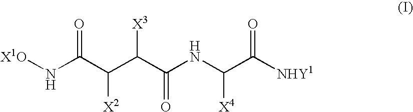
where:
X¹ is H, C₁₋₃ alkyl or C₁₋₃ fluoroalkyl;
X² is H, C₁₋₆ alkyl, C₃₋₆ cycloalkyl or C₁₋₆ fluoroalkyl;
X³ is an X² group, NH₂, C₁₋₁₀ amino or —NH(CO)Xa where Xa is C₁₋₆ alkyl, C₃₋₁₂ aryl or C₅₋₁₅ aralkyl;
X⁴ is C₁₋₆ alkyl, Ar¹ or —(C₁₋₃ alkyl)Ar¹, where Ar¹ is a C₃₋₁₂ aryl or heteroaryl group or —(CH₂)_{w}CONHY², where w is an integer of value 1 or 2;
Y¹ and Y² are independently Y groups, where Y is C₁₋₁₀ alkyl, C₃₋₁₀ cycloalkyl, C₁₋₁₀ fluoroalkyl, an Ar¹ group or —(C₁₋₃ alkyl)Ar¹;
with the provisos that:
(i) X² and X³ are not both H;
(ii) when X¹ is H, X² is H or C₁₋₃ alkyl and X³ is C₁₋₆ alkyl, C₃₋₆ cycloalkyl or C₁₋₆ fluoroalkyl, and X⁴ is C₁₋₆ alkyl, phenyl or benzyl, the imaging moiety does not comprise a chelating agent.

2. The imaging agent of claim 1, where X¹ is H.

3. The imaging agent of claim 1, where X² or X³ is C₁₋₄ alkyl.

4. The imaging agent of claim 3, where X³ is an X² group.

5. The imaging agent of claim 1, wherein X⁴ is —CH₂Ar¹¹.

6. The imaging agent of claim 5, wherein X⁴ comprises an indole group.

7. The imaging agent of claim 1, which is of Formula II: 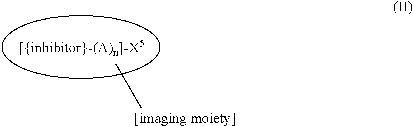
where:
{inhibitor} is the metalloproteinase inhibitor of Formula (I) of claim 1;
-(A)ₙ- is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
R is independently chosen from H, C₁₋₄ alkyl, C₂₋₄ alkenyl, C₂₋₄ alkynyl, C₁₋₄ alkoxyalkyl or C₁₋₄ hydroxyalkyl;
n is an integer of value 0 to 10; and
X⁵ is H, OH, C₁₋₄ alkyl, C₁₋₄ alkoxy, C₁₋₄ alkoxyalkyl, C₁₋₄ hydroxyalkyl or
an Ar¹ group as defined in claim 1.

8. The imaging agent of claim 7, where the imaging moiety is attached at the X⁴ or Y¹ positions of the metalloproteinase inhibitor.

9. The imaging agent of claim 1, where the imaging moiety is chosen from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging;
(vii) a β-emitter suitable for intravascular detection.

10. The imaging agent of claim 1, where the matrix metalloproteinase inhibitor is conjugated to a ligand, and said ligand forms a metal complex with the radioactive metal ion or paramagnetic metal ion.

11. The imaging agent of claim 10, where the ligand is a chelating agent.

12. The imaging agent of claim 10, where the radioactive metal ion is a gamma emitter or a positron emitter.

13. The imaging agent of claim 12, where the radioactive metal ion is ^{99m}Tc, ¹¹¹In, ⁶⁴Cu, ⁶⁷Cu, ⁶⁷Ga or ⁶⁸Ga.

14. The imaging agent of claim 9, where the gamma-emitting radioactive halogen imaging moiety is ¹²³I.

15. The imaging agent of claim 9, where the positron-emitting radioactive non-metal is chosen from ¹⁸F, ¹¹C, ¹³N or ¹²⁴I.

16. The imaging agent of claim 1, where the matrix metalloproteinase inhibitor is of Formula IV: 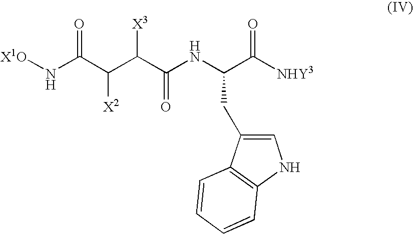
where:
X¹, X² and X³ are as defined in claim 1;
Y³ is a Y group as defined in claim 1.

17. The imaging agent of claim 1, where the matrix metalloproteinase inhibitor is of Formula V: 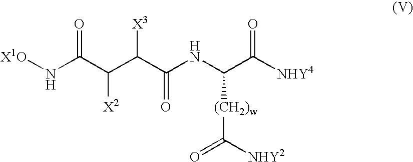
where:
X¹, X³, Y² and w are as defined in claim 1;
Y⁴ is a Y group as defined in claim 1.

18. A pharmaceutical composition which comprises the imaging agent of claim 1 together with a biocompatible carrier, in a form suitable for mammalian administration.

19. A radiopharmaceutical composition which comprises the imaging agent of claim 1 wherein the imaging moiety is radioactive, together with a biocompatible carrier, in a form suitable for mammalian administration.

20. The radiopharmaceutical composition of claim 19, where the imaging moiety comprises a radioactive metal ion.

21. The radiopharmaceutical composition of claim 19, where the imaging moiety comprises a positron-emitting radioactive non-metal or a gamma-emitting radioactive halogen.

22. A conjugate of a matrix metalloproteinase inhibitor of Formula (I) as defined in claim 1 with a ligand, wherein said ligand is capable of forming a metal complex with a radioactive or paramagnetic metal ion.

23. The conjugate of claim 22, of Formula IIb: 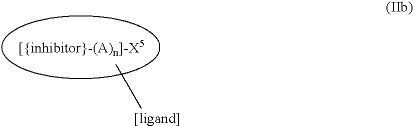
where {inhibitor} is the metalloproteinase inhibitor of Formula (I) 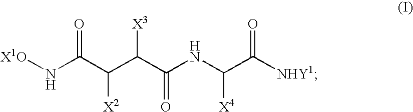
-(A)ₙ- is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
n is an integer of value 0 to 10; and
X⁵ is H, OH, C₁₋₄ alkyl, C₁₋₄ alkoxy, C₁₋₄ alkoxyalkyl, C₁₋₄ hydroxyalkyl or an Ar¹ group as defined in claim 1.

24. The conjugate of claim 22, wherein the matrix metalloproteinase inhibitor is of Formulae IV 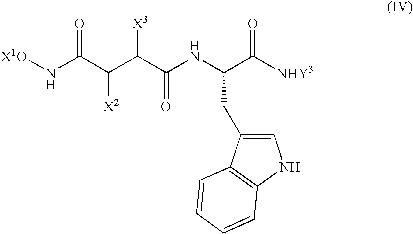
where:
X¹ is H, C₁₋₃ alkyl or C₁₋₃ fluoroalkyl;
X² is H, C₁₋₆ alkyl, C₃₋₆ cycloalkyl or C₁₋₆ fluoroalkyl;
X³ is an X² group, NH₂, C₁₋₁₀ amino or —NH(CO)X^{a} where X^{a} is C₁₋₆ alkyl, C₃₋₁₂ aryl or C₅₋₁₅ aralkyl;
Y³ is C₁₋₁₀ alkyl, C₃₋₁₀ cycloalkyl, C₁₋₁₀ fluoroalkyl, an Ar¹ group or —(C₁₋₃ alkyl)Ar¹; or 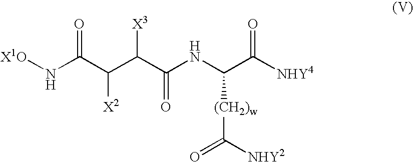
where:
X¹ is H, C₁₋₃ alkyl or C₁₋₃ fluoroalkyl;
X² is H, C₁₋₆ alkyl, C₃₋₆ cycloalkyl or C₁₋₆ fluoroalkyl;
X¹ is an X² group, NH₂, C₁₋₁₀ amino or —NH(CO)X¹ where X¹ is C₁₋₆ alkyl, C₃₋₁₂ aryl or C₁₋₁₅ aralkyl;
w is an integer of value 1 or 2;
Y² are independently Y groups, where Y is C₁₋₁₀ alkyl, C₃₋₁₀ cycloalkyl, C₁₋₁₀ fluoroalkyl, an Ar¹ group or —(C₁₋₃ alkyl)Ar¹;
with the provisos that:
X¹ and X¹ are not both H;
when X¹ is H, X² is H or C₁₋₃ alkyl and X¹ is C₁₋₆ alkyl, C₃₋₆ cycloalkyl or C₁₋₆ fluoroalkyl, and X¹ is C₁₋₆ alkyl, phenyl or benzyl, the imaging moiety does not comprise a chelating agent;
Y⁴ is C₁₋₁₀ alkyl, C₃₋₁₀ cycloalkyl, C₁₋₁₀ fluoroalkyl, an Ar¹ group or —(C₁₋₃ alkyl)Ar¹.

25. The conjugate of claim 22, wherein the ligand is a chelating agent.

26. A precursor for the preparation of the radiopharmaceutical composition of claim 21, which comprises a non-radioactive derivative of the matrix metalloproteinase inhibitor of Formulae (I), wherein said non-radioactive derivative is capable of reaction with a source of the positron-emitting radioactive non-metal or gamma-emitting radioactive halogen to give the desired radiopharmaceutical.

27. The precursor of claim 26, where the source of the positron-emitting radioactive non-metal or gamma-emitting radioactive halogen is chosen from:
(i) halide ion or F⁺ or I⁺; or
(ii) an alkylating agent chosen from an alkyl or fluoroalkyl halide, tosylate, triflate or mesylate.

28. The precursor of claim 26, where the non-radioactive derivative is chosen from:
(i) an organometallic derivative such as a trialkylstannane or a trialkylsilane;
(ii) a derivative containing an alkyl halide, alkyl tosylate or alkyl mesylate for nucleophilic substitution;
(iii) a derivative containing an aromatic ring activated towards nucleophilic or electrophilic substitution;
(iv) a derivative containing a functional group which undergoes facile alkylation;
(v) a derivative which alkylates thiol-containing compounds to give a thioether-containing product.

29. A kit for the preparation of the radiopharmaceutical composition of claim 20, which comprises the conjugate of a matrix metalloproteinase inhibitor of Formula (I) with a ligand, wherein said ligand is capable of forming a metal complex with a radioactive or paramagnetic metal ion.

30. The kit of claim 29, where the radioactive metal ion is ^{99m}Tc, and the kit further comprises a biocompatible reductant.

31. A kit for the preparation of the radiopharmaceutical composition of claim 21, which comprises the precursor for the preparation of the radiopharmaceutical composition, which comprises a non-radioactive derivative of the matrix metalloproteinase inhibitor of Formulae (I), wherein said non-radioactive derivative is capable of reaction with a source of the positron-emitting radioactive non-metal or gamma-emitting radioactive halogen to give the desired radiopharmaceutical.

32. The kit of claim 31, where the precursor is bound to a solid phase.

33. Use of the imaging agent of claim 1 for the diagnostic imaging of atherosclerosis.

34. Use of the imaging agent of claim 1 for the diagnostic imaging of unstable plaques.

35. Use of the imaging agent of claim 1 for the intravascular detection of atherosclerosis.
